(19) United States
(12) Patent Application Publication
(10) Pub. No.: US 2021/0308208 A1
(43) Pub. Date: Oct. 7, 2021

(54) ANTI-TISSUE FACTOR ANTIBODY-DRUG CONJUGATES AND THEIR USE IN THE TREATMENT OF CANCER

(71) Applicant: Genmab A/S, Copenhagen V (DK)

(72) Inventors: Reshma A. RANGWALA, Phildelphia, PA (US); Esther C.W. BREIJ, Driebergen (NL); Sandra VERPLOEGEN, Nieuwegein (NL); Bart DE GOEIJ, Utrecht (NL); Oyewale O. ABIDOYE, Bellevue, WA (US); Leonardo V. NICACIO, Redmond, WA (US); Stephen C. ALLEY, Bothell, WA (US)

(21) Appl. No.: 17/268,387

(22) PCT Filed: Aug. 14, 2019
(86) PCT No.: PCT/US2019/046467

§ 371 Date: Feb. 12, 2021

Related U.S. Application Data
(60) Provisional application No. 62/765,093, filed on Aug. 16, 2018.

(51) Int. Cl. A61K 38/07; A61K 47/68; A61K 47/65; A61K 47/54; A61P 35/00

(52) U.S. Cl. CPC A61K 38/07; A61K 47/6849; A61P 35/00; A61K 47/545; A61K 47/65

(57) ABSTRACT

The invention provides methods and compositions for treating cancer, such as ovarian cancer, peritoneal cancer, and fallopian tube cancer, in a subject, such as by the administration of antibody-drug conjugates that bind to tissue factor (TF). The invention also provides articles of manufacture and compositions comprising said antibody drug-conjugates that bind to TF for use in treating cancer (e.g., ovarian cancer, peritoneal cancer, and fallopian tube cancer).

### CROSS-REFERENCE TO RELATED APPLICATION

This application claims priority to U.S. Provisional application No. 62/765,093 filed on Aug. 16, 2018, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682000940SEQLIST.TXT, date recorded: Aug. 13, 2019, size: 6 KB).

### TECHNICAL FIELD

The present invention relates to anti-tissue factor (TF) antibody-drug conjugates and methods of using the same to treat cancer, such as ovarian cancer, peritoneal cancer, and fallopian tube cancer.

### BACKGROUND

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascade, and it functions as the high-affinity receptor for the coagulation factor VIIa (FVIIa), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, and is associated with more aggressive disease. Furthermore, human TF also exists in a soluble alternatively-spliced form, asHTF. It has been found that asHTF promotes tumor growth (Hobbs et al., 2007, Thrombosis Res. 120(2):S13-S21).

The most common type of ovarian cancer is epithelial ovarian cancer. There are a variety of types of epithelial ovarian cancers, including serous, mucinous, endometroid, clear cell, and others. The stages and treatment are the same for epithelial ovarian, fallopian tube, and primary peritoneal cancers. Platinum doublets are standard of care in first-line advanced ovarian cancer. Almost all patients with advanced disease will receive initial treatment with chemotherapy, and a median overall survival (OS) of nearly four years can be achieved in patients treated with carboplatin plus paclitaxel. Despite survival outcomes that appear better than many other advanced tumor types, in reality this disease is typically characterized by multiple relapses and numerous lines of chemotherapy. The greatest unmet need in ovarian cancer is therapy for patients who arc resistant to or cannot tolerate platinum-based therapy. These patients have very few treatment options. Single-agent therapies used to treat this subset of patients include paclitaxel, pegylated liposomal doxorubicin (PLD) and topotecan. Response rate is in the 10-15% range and overall survival is approximately 12 months. In 2014, FDA approved avastin (bevacizumab) in combination with paclitaxel, PLD or topotecan as treatment for this subset of patients. The combination of avastin with chemotherapy enhanced the progression-free survival (PFS) time from 3.4 months for chemotherapy alone to 6.8 months. Clinical benefit, as measured by PFS and overall survival (OS), diminishes significantly below even the poor prognosis of first line treatment as the line of therapy increases. Thus, there is an urgent need for more effective therapies for the treatment of platinum-resistant ovarian cancer (PROC).

The present invention meets the need for improved treatment of ovarian cancer, peritoneal cancer, and fallopian tube cancer by providing highly specific and effective anti-TF antibody-drug conjugates.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating ovarian cancer, peritoneal cancer or fallopian tube cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.65 mg/kg to about 2.1 mg/kg. In some embodiments, the dose is about 2.0 mg/kg. In some embodiments, the dose is 2.0 mg/kg. In some of any of the embodiments herein, the antibody-drug conjugate is administered once about every 3 weeks. In some of any of the embodiments herein, the antibody-drug conjugate is administered once every 3 weeks. In some of any of the embodiments herein, the dose is about 0.65 mg/kg. In some of any of the embodiments herein, the dose is 0.65 mg/kg. In some of any of the embodiments herein, the dose is about 0.9 mg/kg. In some of any of the embodiments herein, the dose is 0.9 mg/kg. In some of any of the embodiments herein, the antibody-drug conjugate is administered once every week. In some of any of the embodiments herein, the antibody-drug conjugate is administered once about every week. In some of any of the embodiments herein, the antibody-drug conjugate is administered once about every 1 week for 3 consecutive weeks followed by about a 1 week resting period during which the antibody-drug conjugate is not administered. In some of any of the embodiments herein, the antibody-drug conjugate is administered once every 1 week for three consecutive weeks followed by a one week resting period during which the antibody-drug conjugate is not administered. In some of any of the embodiments herein, the antibody-drug conjugate is administered on about days 1, 8, and 15 of about a 4-week cycle. In some of any of the embodiments herein, the antibody-drug conjugate is administered on days 1, 8, and 15 of a 4-week cycle. In some of any of the embodiments herein, the subject has been previously treated with one or more therapeutic agents and did not respond to the treatment, wherein the one or more therapeutic agents is not the antibody-drug conjugate. In some of any of the embodiments herein, the subject has been previously treated with one or more therapeutic agents and relapsed after the treatment, wherein the one or more therapeutic agents is not the antibody-drug conjugate. In some of any of the embodiments herein, the subject has been previously treated with one or more therapeutic agents and has experienced disease progression during treatment, wherein the one or more therapeutic agents is not the antibody-drug conjugate. In some of any of the embodiments herein, the subject has been previously treated with a platinum-based therapy. In some of any of the embodiments herein, the cancer is platinum-resistant. In some of any of the embodiments herein, the subject experienced disease progression or relapsed 2 or more months after treatment with the platinum-based therapy. In some of any of the embodiments herein, the subject experienced disease progression or relapsed within 6 months after treatment with the platinum-based therapy. In some of any of the embodiments herein, the subject experienced disease progression or relapsed between 2 months and 6 months after treatment with the platinum-based therapy. In some of any of the embodiments herein, cancer is not platinum-refractory. In some of any of the embodiments herein, the subject did not experience disease progression or relapse within 2 months after treatment with the platinum-based therapy. In some of any of the embodiments herein, the subject has been previously treated with a VEGF antagonist. In some of any of the embodiments herein, the VEGF antagonist is an anti-VEGF antibody. In some of any of the embodiments herein, the anti-VEGF antibody is bevacizumab. In some of any of the embodiments herein, the subject received prior systemic therapy and experienced disease progression on or after the systemic therapy. In some of any of the embodiments herein, the subject received 1, 2, 3, 4 or 5 rounds of prior systemic therapy. In some of any of the embodiments herein, the rounds of prior systemic therapy are in the platinum resistant setting. In some of any of the embodiments herein, the prior systemic therapy is a chemotherapy regimen and wherein poly ADP ribose polymerase (PARP) inhibitors are not chemotherapy. In some of any of the embodiments herein, the cancer is ovarian cancer. In some of any of the embodiments herein, the ovarian cancer is epithelial ovarian cancer. In some of any of the embodiments herein, the cancer is peritoneal cancer. In some of any of the embodiments herein, the peritoneal cancer is primary peritoneal cancer. In some of any of the embodiments herein, the cancer is fallopian tube cancer. In some of any of the embodiments herein, the cancer is an advanced stage cancer. In some of any of the embodiments herein, the advanced stage cancer is a stage 3 or stage 4 cancer. In some of any of the embodiments herein, the advanced stage cancer is metastatic cancer. In some of any of the embodiments herein, the cancer is recurrent cancer. In some of any of the embodiments herein, the monomethyl auristatin is monomethyl auristatin E (MMAE). In some of any of the embodiments herein, the anti-TF antibody or antigen-binding fragment thereof of the antibody-drug conjugate is a monoclonal antibody or a monoclonal antigen-binding fragment thereof. In some of any of the embodiments herein, the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

In some of any of the embodiments herein, the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8. In some of any of the embodiments herein, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some of any of the embodiments herein, the anti-TF antibody of the antibody-drug conjugate is tisotumab. In some of any of the embodiments herein, the antibody-drug conjugate further comprises a linker between the anti-TF antibody or antigen-binding fragment thereof and the monomethyl auristatin. In some of any of the embodiments herein, the linker is a cleavable peptide linker. In some of any of the embodiments herein, the cleavable peptide linker has a formula: -MC-vc-PAB-, wherein:

a) MC is:

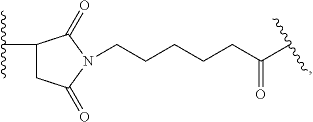 [SMILES: *C(=O)CCCCCN1C(=O)CC(*)C1=O]

b) vc is the dipeptide valine-citrulline, and

c) PAB is:

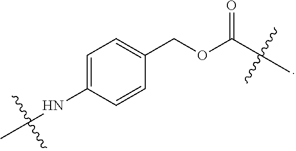 [SMILES: *Nc1ccc(COC(*)=O)cc1]

In some of any of the embodiments herein, In some of any of the embodiments herein, the linker is attached to sulphydryl residues of the anti-TF antibody obtained by partial reduction or full reduction of the anti-TF antibody or antigen-binding fragment thereof. In some of any of the embodiments herein, the linker is attached to monomethyl auristatin E (MMAE), wherein the antibody-drug conjugate has the following structure:

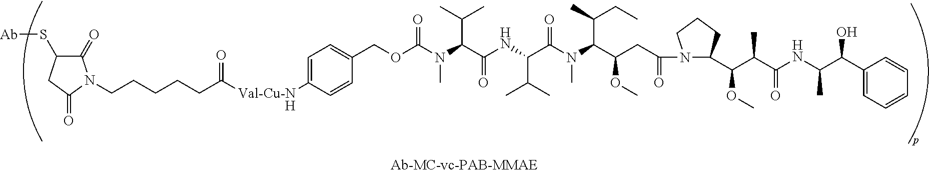

wherein p denotes a number from 1 to 8, S represents a sulphydryl residue of the anti-TF antibody, and Ab designates the anti-TF antibody or antigen-binding fragment thereof. In some of any of the embodiments herein, the average value of p in a population of the antibody-drug conjugates is about 4. In some of any of the embodiments herein, the antibody-drug conjugate is tisotumab vedotin. In some of any of the embodiments herein, the route of administration for the antibody-drug conjugate is intravenous. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF. In some of any of the embodiments herein, one or more therapeutic effects in the subject is improved after administration of the antibody-drug conjugate relative to a baseline. In some of any of the embodiments herein, the one or more therapeutic effects is selected from the group consisting of: size of a tumor derived from the cancer, objective response rate, duration of response, time to response, progression free survival, overall survival and CA-125 level. In some of any of the embodiments herein, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate. In some of any of the embodiments herein, the objective response rate is at least about 20%, at least about 25%, at least about 30%, at least about 35%, at least about 40%, at least about 45%, at least about 50%, at least about 60%, at least about 70%, or at least about 80%. In some of any of the embodiments herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample obtained from the subject before administration of the antibody-drug conjugate. In some of any of the embodiments herein, the subject has one or more adverse events and is further administered an additional therapeutic agent to eliminate or reduce the severity of the one or more adverse events. In some of any of the embodiments herein, the subject is at risk of developing one or more adverse events and is further administered an additional therapeutic agent to prevent or reduce the severity of the one or more adverse events. In some of any of the embodiments herein, the one or more adverse events is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, or general physical health deterioration. In some of any of the embodiments herein, the one or more adverse events is a grade 3 or greater adverse event. In some of any of the embodiments herein, the one or more adverse events is a serious adverse event. In some of any of the embodiments herein, the one or more adverse events is conjunctivitis and/or keratitis and the additional agent is a preservative-free lubricating eye drop, an ocular vasoconstrictor and/or a steroid eye drop. In some of any of the embodiments herein, the antibody-drug conjugate is administered as a monothcrapy. In some of any of the embodiments herein, the subject is a human. In some of any of the embodiments herein, the antibody-drug conjugate is in a pharmaceutical composition comprising the antibody-drug conjugate and a pharmaceutical acceptable carrier.

Also provided herein are kits comprising:

(a) a dosage ranging from about 0.65 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and

(b) instructions for using the antibody drug conjugate according to the method of any one of embodiments herein.

### BRIEF DESCRIPTION OF THE DRAWINGS

FIG. 1 is a diagram showing the mechanism of action (MOA) of the antibody-drug conjugate tisotumab vedotin.

FIG. 2 shows an overview of the study design of a clinical trial of tisotumab vedotin administered once every three weeks (Q3W regimen) or on days 1, 8, and 15 of each 28-day cycle (dose dense regimen).

FIG. 3 shows in vivo anti-tumor activity of tisotumab vedotin in a SKOV-3 xenograft model in SCID mice. Average tumor size in the SKOV-3 xenograft model in SCID mice after treatment with tisotumab vedotin (1 or 4 mg/kg), an isotype control ADC (IgG1-b12-MMAE, 4 mg/kg) or an isotype control IgG (IgG1-b12, 4 mg/kg). Tumor size was assessed by caliper measurement. Error bars indicate standard error of the mean (S.E.M.).

FIG. 4 shows in vivo anti-tumor activity of tisotumab vedotin in an ovarian cancer patient-derived xenograft model in nude mice. Average tumor size in the OVFX 1993 patient-derived xenograft model in athymic nude mice after treatment with tisotumab vedotin (4 mg/kg), an isotype control ADC (IgG1-b12-MMAE, 4 mg/kg) or an isotype control IgG (IgG1-b12, 4 mg/kg). Tumor size was assessed by caliper measurement. Error bars indicate standard error of the mean (S.E.M.).

FIG. 5A shows in vivo anti-tumor activity of tisotumab vedotin in an ovarian cancer patient-derived xenograft model in nude mice. Average tumor size in the CTG-0956 patient-derived xenograft model in athymic nude mice after treatment with tisotumab vedotin (2 mg/kg) or an isotype control (2 mg/kg). Tumor size was assessed by caliper measurement. Error bars indicate standard error of the mean (S.E.M.). FIG. 5B shows the weight of the mice after treatment with tisotumab vedotin (2 mg/kg) or an isotype control (2 mg/kg).

FIG. 6A shows in vivo anti-tumor activity of tisotumab vedotin in an ovarian cancer patient-derived xenograft model in nude mice. Average tumor size in the CTG-1086 patient-derived xenograft model in athymic nude mice after treatment with tisotumab vedotin (2 mg/kg) or an isotype control (2 mg/kg). Tumor size was assessed by caliper measurement. Error bars indicate standard error of the mean (S.E.M.). FIG. 6B shows the weight of the mice after treatment with tisotumab vedotin (2 mg/kg) or an isotype control (2 mg/kg).

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A. B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press: The Dictionary of Cell and Molecular Biology, 3rd ed., 1999, Academic Press; and the Oxford Dictionary Of Biochemistry And Molecular Biology. Revised, 2000, Oxford University Press, provide one of skill with a general dictionary of many of the terms used in this disclosure.

Units, prefixes, and symbols are denoted in their Système Intemational de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. In some embodiments, tissue factor comprises the amino acid sequence found under Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. Unless stated otherwise, the numbering of amino acid residues in the constant region is according to the EU-index as described in Kabat et al., Sequences of Proteins of Immunological Interest, 5th Ed. Public Health Service, National Institutes of Health, Bethesda, Md. (1991). An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1. CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, cascin) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-TF antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

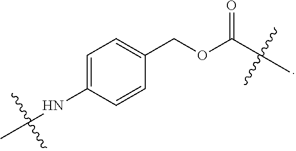 [SMILES: *Nc1ccc(COC(*)=O)cc1]

The abbreviation “MC” refers to the stretcher maleimidocaproyl:

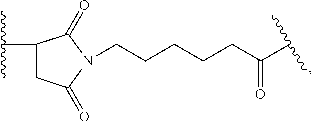 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

A “platinum-based therapy” refers to treatment with a platinum-based agent. A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future. Platinum-based therapies may include, but are not limited to, carboplatin, cisplatin, oxaliplatin, nedaplatin, triplatin tetranitrate, phenanthriplatin, picoplatin and satraplatin.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

A therapeutically effective amount of a drug (e.g., an anti-TF antibody-drug conjugate) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-TF antibody-drug conjugate) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD (progressive disease), taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein. “overall survival” or “OS” refers to the percentage of individuals in a group who are likely to be alive after a particular duration of time.

The term “weight-based dose”, as referred to herein, means that a dose administered to a patient is calculated based on the weight of the patient. For example, when a patient with 60 kg body weight requires 2 mg/kg of an anti-TF antibody-drug conjugate, one can calculate and use the appropriate amount of the anti-TF antibody-drug conjugate (i.e., 120 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-TF antibody-drug conjugate). For example, a 60 kg person and a 100 kg person would receive the same dose of an antibody-drug conjugate (e.g., 240 mg of an anti-TF antibody-drug conjugate).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isocrotonate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” refers to the physical introduction of a therapeutic agent to a subject, using any of the various methods and delivery systems known to those skilled in the art. Exemplary routes of administration for the anti-TF antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “baseline” or “baseline value” used interchangeably herein can refer to a measurement or characterization of a symptom before the administration of the therapy (e.g., an antibody-drug conjugate as described herein) or at the beginning of administration of the therapy. The baseline value can be compared to a reference value in order to determine the reduction or improvement of a symptom of a TF-associated disease contemplated herein (e.g., ovarian cancer, peritoneal cancer, or fallopian tube cancer). The terms “reference” or “reference value” used interchangeably herein can refer to a measurement or characterization of a symptom after administration of the therapy (e.g., an antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value; a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the antibody drug conjugate is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically important”
Requires inpatient hospitalization or prolongation of existing hospitalization, excluding the following: 1) routine treatment or monitoring of the underlying disease, not associated with any deterioration in condition, 2) elective or pre-planned treatment for a pre-existing condition that is unrelated to the indication under study and has not worsened since signing the informed consent, and social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively. “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Antibody-Drug Conjugates

The present invention provides an anti-TF antibody-drug conjugate that binds to TF for use in the treatment of ovarian cancer, peritoneal cancer, or fallopian tube cancer in a subject, wherein the antibody-drug conjugate comprises an anti-TF antibody or an antigen-binding fragment thereof conjugated to a monomethyl auristatin or a functional analog thereof or a functional derivative thereof. In some embodiments, the cancer is ovarian cancer. In some embodiments, the ovarian cancer is epithelial ovarian cancer. In some embodiments, the cancer is peritoneal cancer. In some embodiments, the peritoneal cancer is primary peritoneal cancer. In some embodiments, the cancer is fallopian tube cancer. In some embodiments, the ovarian cancer, peritoneal cancer, or fallopian tube cancer is a metastatic cancer. In some embodiments, the subject has relapsed, recurrent and/or metastatic ovarian cancer, peritoneal cancer, or fallopian tube cancer. In some embodiments, the subject has been previously treated with a platinum-based therapy. In some embodiments, the cancer is platinum-resistant, wherein platinum-resistant cancer means that the subject experienced disease progression or relapsed between 2 months and 6 months after treatment with the platinum-based therapy. In some embodiments, the cancer is not platinum-refractory, wherein platinum-refractory means that the subject experienced disease progression or relapse within 2 months after treatment with the platinum-based therapy.

A. Anti-TF Antibody

Generally, anti-TF antibodies of the disclosure bind TF, e.g., human TF, and exert cytostatic and cytotoxic effects on malignant cells, such as ovarian cancer cells. Anti-TF antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies are antigen-binding fragments (e.g., human antigen-binding fragments) as described herein and include, but are not limited to, Fab, Fab′ and F(ab′)₂. Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken.

The anti-TF antibodies of the present disclosure may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme): and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

CDR sequences provided herein are according to the IMGT method as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, anti-TF antibodies that compete with tisotumab binding to TF are also provided herein. Anti-TF antibodies that bind to the same epitope as tisotumab are also provided herein.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO:13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of EVQLLESGGGLVQPGGSLRLSCAASGFTFSNYAMSWVRQAPGKGLEWVSSISGSGDYT YYTDSVKGRFTISRDNSKNTLYLQMNSLRAEDTAVYYCARSPWGYYLDSWGQGTLVT VSS (SEQ ID NO:7) and the light chain variable domain comprises the amino acid sequence of

(SEQ ID NO: 8)
DIQMTQSPPSLSASAGDRVTITCRASQGISSRLAWYQQKPEKAPKSLIYAA
SSLQSGVPSRFSGSGSGTDFTLTISSLQPEDFATYYCQQYNSYPYTFGQGT
KLEIK.

In some embodiments of the anti-TF antibodies described herein, the heavy chain CDR sequences comprise the following:

a) CDR-H1
(SEQ ID NO: 1)
(GFTFSNYA);
b) CDR-H2
(SEQ ID NO: 2)
(ISGSGDYT);
and
c) CDR-H3
(SEQ ID NO: 3)
(ARSPWGYYLDS).

In some embodiments of the anti-TF antibodies described herein, the heavy chain FR sequences comprise the following:

a) HC-FR1
(SEQ ID NO: 9)
(EVQLLESGGGLVQPGGSLRLSCAAS);
b) HC-FR2
(SEQ ID NO: 10)
(MSWVRQAPGKGLEWVSS);
c) HC-FR3
(SEQ ID NO: 11)
(YYTDSVKGRFTISRDNSKNTLYLQMNSLRAEDTAVYYC);
and
d) HC-FR4
(SEQ ID NO: 12)
(WGQGTLVTVSS).

In some embodiments of the anti-TF antibodies described herein, the light chain CDR sequences comprise the following:

a) CDR-L1
(SEQ ID NO: 4)
(QGISSR);
b) CDR-L2
(SEQ ID NO: 5)
(AAS);
and
c) CDR-L3
(SEQ ID NO: 6)
(QQYNSYPYT).

In some embodiments of the anti-TF antibodies described herein, the light chain FR sequences comprise the following:

a) LC-FR1
(SEQ ID NO: 13)
(DIQMTQSPPSLSASAGDRVTITCRAS);
b) LC-FR2
(SEQ ID NO: 14)
(LAWYQQKPEKAPKSLIY);
c) LC-FR3
(SEQ ID NO: 15)
(SLQSGVPSRFSGSGSGTDFTLTISSLQPEDFATYYC);
and
d) LC-FR4
(SEQ ID NO: 16)
(FGQGTKLEIK).

In some embodiments, provided herein is an anti-TF antibody that binds to TF (e.g., human TF), wherein the antibody comprises a heavy chain variable region and a light chain variable region, wherein the antibody comprises:

(a) heavy chain variable domain comprising:
(1) an HC-FR1 comprising the amino acid sequence of SEQ ID NO:9;
(2) an CDR-H1 comprising the amino acid sequence of SEQ ID NO: 1;
(3) an HC-FR2 comprising the amino acid sequence of SEQ ID NO: 10;
(4) an CDR-H2 comprising the amino acid sequence of SEQ ID NO: 2;
(5) an HC-FR3 comprising the amino acid sequence of SEQ ID NO: 11.
(6) an CDR-H3 comprising the amino acid sequence of SEQ ID NO:3; and
(7) an HC-FR4 comprising the amino acid sequence of SEQ ID NO:12, and/or

(b) a light chain variable domain comprising:
(1) an LC-FR1 comprising the amino acid sequence of SEQ ID NO:13;
(2) an CDR-L1 comprising the amino acid sequence of SEQ ID NO:4;
(3) an LC-FR2 comprising the amino acid sequence of SEQ ID NO:14;
(4) an CDR-L2 comprising the amino acid sequence of SEQ ID NO:5;
(5) an LC-FR3 comprising the amino acid sequence of SEQ ID NO:15;
(6) an CDR-L3 comprising the amino acid sequence of SEQ ID NO:6; and
(7) an LC-FR4 comprising the amino acid sequence of SEQ ID NO:16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the present invention may also be described or specified in terms of their binding affinity to TF (e.g., human TF). Preferred binding affinities include those with a dissociation constant or K_{D} less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

B. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzyl carbamate (PAB). In some embodiments, the cleavable peptide linker has the formula: MC-vc-PAB-, wherein:

a) MC is:

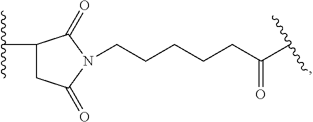 [SMILES: *C(=O)CCCCCN1C(=O)CC(*)C1=O]

b) vc is the dipeptide valine-citrulline, and

c) PAB is:

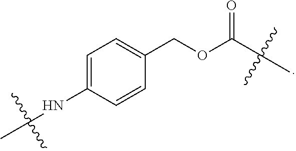 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:

a) MC is:

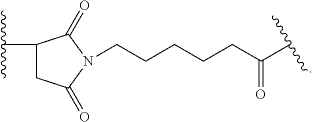 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. No. 5,663,149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

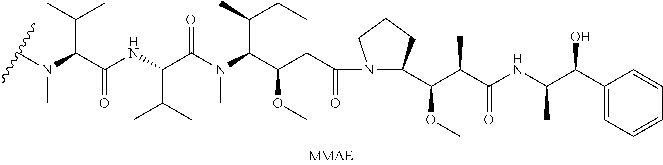 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

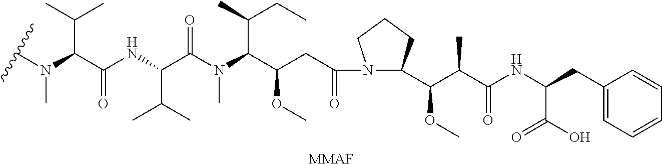

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

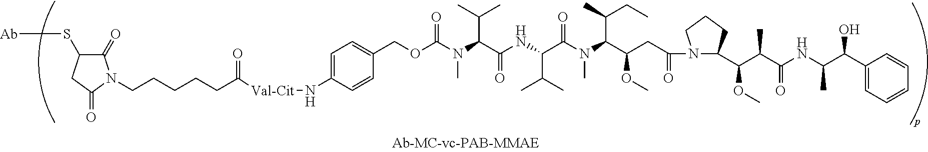

wherein p denotes a number from 1 to 8, e.g., 1, 2, 3, 4, 5, 6, 7 or 8, e.g., p may be from 3-5, S represents a sulphydryl residue of the anti-TF antibody and Ab designates a anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. In another embodiment, a non-cleavable linker MC is attached to MMAF. The resulting linker-auristatin MC-MMAF is also designated mcMMAF. Both the vcMMAF and mcMMAF drug linker moieties and conjugation methods are disclosed in WO2005081711 and U.S. Pat. No. 7,498,298. When vcMMAF or mcMMAF is attached to an anti-TF antibody or antigen-binding fragment thereof as described herein, the resulting structure is:

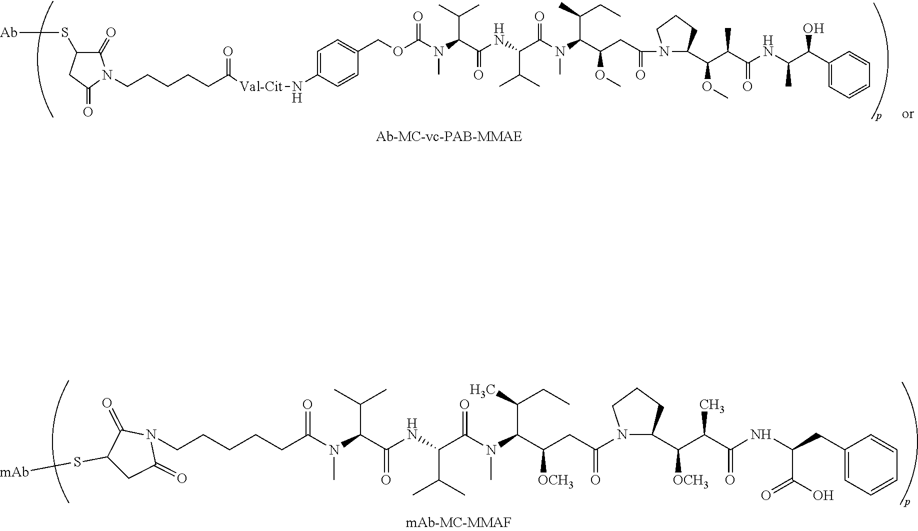

wherein p denotes a number from 1 to 8, e.g., 1, 2, 3, 4, 5, 6, 7 or 8, e.g., p may be from 3-5, S represents a sulphydryl residue of the anti-TF antibody and Ab or mAb designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

C. Nucleic Acids, Host Cells and Methods of Production

In some aspects, also provided herein are nucleic acids encoding an anti-TF antibody or antigen-binding fragment thereof as described herein. Further provided herein are vectors comprising the nucleic acids encoding an anti-TF antibody or antigen-binding fragment thereof as described herein. Further provided herein are host cells expressing the nucleic acids encoding an anti-TF antibody or antigen-binding fragment thereof as described herein. Further provided herein are host cells comprising the vectors comprising the nucleic acids encoding an anti-TF antibody or antigen-binding fragment thereof as described herein. Methods of producing an anti-TF antibody, linker and anti-TF antibody-drug conjugate are described in U.S. Pat. No. 9,168,314.

The anti-TF antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190. EP216846, U.S. Pat. No. 5,981,216, WO 87/04462, EP323997, U.S. Pat. Nos. 5,591,639, 5,658,759, EP338841, U.S. Pat. No. 5,879,936, and 5,891,693.

After isolating and purifying the anti-TF antibodies from the cell media using well known techniques in the art, they are conjugated with an auristatin via a linker as described in U.S. Pat. No. 9,168,314.

Monoclonal anti-TF antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature. 352, 624-628 (1991) and Marks et al., J Mol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-TF antibody) of the invention is a human antibody. Human monoclonal antibodies directed against TF may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and a light chain immunoglobulin sequences, together with targeted mutations that inactivate the endogenous p and a chain loci (Lonberg, N. et al., Nature, 368, 856-859 (1994)). Accordingly, the mice exhibit reduced expression of mouse IgM or K and in response to immunization, the introduced human heavy and light chain transgenes undergo class switching and somatic mutation to generate high affinity human IgG,κ monoclonal antibodies (Lonberg. N. et al. (1994), supra; reviewed in Lonberg, N. Handbook of Experimental Pharmacology 113, 49-101 (1994). Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology. 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585. WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a ˜460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain transgenes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology. 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KCO05 [MIK] (Balb) mice which were generated by backcrossing the KC05 strain (as described in Fishwild et al. (1996) Nature Biotechnology. 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol. Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990). Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Methods of Treatment

The most common type of ovarian cancer is epithelial ovarian cancer. There are a variety of types of epithelial ovarian cancers, including serous, mucinous, endometroid, clear cell, and others. Cancers of the ovary, fallopian tube and of peritoneal origin in women exhibit similar clinical characteristics and behavior. The stages and treatment are the same for epithelial ovarian, fallopian tube, and primary peritoneal cancers. In 2015, 1.2 million women were estimated to be living with ovarian cancer, and ovarian cancer resulted in 161.100 deaths worldwide. Chemotherapy, typically consisting of platins combined with non-platins, has been a general standard of care for ovarian cancer for decades. Despite initial therapy, the vast majority of women with ovarian cancer will relapse and require subsequent therapy. Patients whose disease relapses within 6 months after platinum-containing therapy are categorized as having platinum-resistant disease. At first relapse, approximately 25% of the patients have platinum-resistant ovarian cancer (PROC), and the vast majority of patients with recurrent disease will eventually develop PROC. For most PROC patients, single agent chemotherapy rather than combination therapy is favored in the first line. Single agents approved for PROC have overall RECIST response rates around 12% and progression-free survival (PFS) around 3.4 months. For patients who relapse after first-line therapy for PROC and are fit enough to receive subsequent treatment there is no standard of care. Clinical benefit, as measured by PFS and overall survival (OS), diminishes significantly below even the poor prognosis of first line treatment as the line of therapy increases.

The invention provides methods for treating cancer in a subject with an anti-TF antibody-drug conjugate described herein, wherein the cancer is ovarian cancer, peritoneal cancer or fallopian tube cancer. In some embodiments, the cancer is ovarian cancer. In some embodiments, the ovarian cancer is epithelial ovarian cancer. In some embodiments, the cancer is peritoneal cancer. In some embodiments, the peritoneal cancer is primary peritoneal cancer. In some embodiments, the cancer is fallopian tube cancer. In some embodiments, the ovarian cancer, peritoneal cancer, or fallopian tube cancer is a metastatic cancer. In some embodiments, the subject has relapsed, recurrent and/or metastatic ovarian cancer, peritoneal cancer, or fallopian tube cancer. In one aspect, the antibody-drug conjugate is tisotumab vedotin. In a particular embodiment, the subject is a human.

In another aspect the present invention provides an antibody-drug conjugate that binds to TF for use in the treatment of cancer wherein the antibody-drug conjugate comprises an anti-TF antibody or an antigen-binding fragment thereof conjugated to a monomethyl auristatin or a functional analog thereof or a functional derivative thereof and wherein the cancer is ovarian cancer, peritoneal cancer or fallopian tube cancer. In some embodiments, the cancer is ovarian cancer. In some embodiments, the ovarian cancer is epithelial ovarian cancer. In some embodiments, the cancer is peritoneal cancer. In some embodiments, the peritoneal cancer is primary peritoneal cancer. In some embodiments, the cancer is fallopian tube cancer. In some embodiments, the ovarian cancer, peritoneal cancer, or fallopian tube cancer is a metastatic cancer. In some embodiments, the subject has relapsed, recurrent and/or metastatic ovarian cancer, peritoneal cancer, or fallopian tube cancer. In one aspect, the antibody-drug conjugate is tisotumab vedotin. In a particular embodiment, the subject is a human.

In some embodiments, the subject has been previously treated for the ovarian cancer, peritoneal cancer or fallopian tube cancer. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-TF antibody-drug conjugate as described herein.

The invention provides methods for treating ovarian cancer, peritoneal cancer or fallopian tube cancer with an antibody-drug conjugate described herein. In one aspect, the antibody-drug conjugates described herein are for use in a method of treating ovarian cancer, peritoneal cancer or fallopian tube cancer in a subject. In one aspect, the antibody-drug conjugate is tisotumab vedotin. In some embodiments, the subject has not been previously treated for the ovarian cancer, peritoneal cancer or fallopian tube cancer. In some embodiments, the cancer is ovarian cancer. In some embodiments, the ovarian cancer is epithelial ovarian cancer. In some embodiments, the cancer is peritoneal cancer. In some embodiments, the peritoneal cancer is primary peritoneal cancer. In some embodiments, the cancer is fallopian tube cancer. In some embodiments, the ovarian cancer, peritoneal cancer, or fallopian tube cancer is a metastatic cancer. In some embodiments, the subject has relapsed, recurrent and/or metastatic ovarian cancer, peritoneal cancer, or fallopian tube cancer. In some embodiments, the subject has received at least one previous treatment for the ovarian cancer, peritoneal cancer or fallopian tube cancer. In some embodiments, the subject received prior systemic therapy for the ovarian cancer, peritoneal cancer or fallopian tube cancer. In some embodiments, the subject experienced disease progression on or after the systemic therapy. In some embodiments, the subject received no more than 5 rounds of prior systemic therapy. In some embodiments, the subject received 1, 2, 3, 4 or 5 rounds of prior systemic therapy. In some embodiments, the subject received 1, 2, 3, 4 or 5 rounds of prior systemic therapy in the platinum resistant setting. In some embodiments, the rounds of prior systemic therapy were for the treatment of platinum-resistant ovarian cancer (PROC). In some embodiments, the subject received 1 round of prior systemic therapy. In some embodiments, the subject received 2 rounds of prior systemic therapy. In some embodiments, the subject received 3 rounds of prior systemic therapy. In some embodiments, the subject received 4 rounds of prior systemic therapy. In some embodiments, the subject received 5 rounds of prior systemic therapy. In some embodiments, the prior systemic therapy is a chemotherapy regimen. In some embodiments, treatment with a poly ADP ribose polymerase (PARP) inhibitor regimen is not a chemotherapy regimen. In some embodiments, the subject has been previously treated with a platinum-based therapy. In some embodiments, the cancer is platinum-resistant, wherein the subject experienced disease progression or relapsed between 2 months and 6 months after treatment with the platinum-based therapy. In some embodiments, the cancer is not platinum-refractory, wherein the subject experienced disease progression or relapse within 2 months after treatment with the platinum-based therapy. In some embodiments, the subject has been previously treated with a VEGF antagonist. In some embodiments, the subject has been previously treated with bevacizumab. In some embodiments, the ovarian cancer, peritoneal cancer or fallopian tube cancer is an advanced stage cancer. In some embodiments, the advanced stage cancer is a stage 3 or 4 cancer. In some embodiments, the advanced stage cancer is a metastatic cancer. In some embodiments, the ovarian cancer, peritoneal cancer or fallopian tube cancer is a recurrent cancer. In a particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the ovarian cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the ovarian cancer, peritoneal cancer or fallopian tube cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

A. Routes of Administration

An anti-TF antibody-drug conjugate or antigen-binding fragment thereof described herein can be administered by any suitable route and mode. Suitable routes of administering antibody-drug conjugate of the present invention are well known in the art and may be selected by those of ordinary skill in the art. In one embodiment, the antibody-drug conjugate is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-TF antibody-drug conjugate or antigen-binding fragment described herein is intravenous injection or infusion. In some embodiments, the route of administration of an anti-TF antibody-drug conjugate or antigen-binding fragment described herein is intravenous infusion.

B. Dosage and Frequency of Administration

In one aspect, the present invention provides for methods of treating a subject with ovarian cancer, peritoneal cancer, or fallopian tube cancer as described herein with a particular dose of an anti-TF antibody-drug conjugate or antigen-binding fragment thereof as described herein, wherein the subject is administered the antibody-drug conjugate or antigen-binding fragment thereof as described herein with a particular frequency.

In one embodiment of the methods or uses or product for uses provided herein, an anti-TF antibody-drug conjugate or antigen-binding fragment thereof as described herein is administered to the subject at a dose ranging from about 0.65 mg/kg to about 2.1 mg/kg of the subject's body weight. In certain embodiments, the dose is about 0.65 mg/kg, about 0.7 mg/kg, about 0.75 mg/kg, about 0.8 mg/kg, about 0.85 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 0.65 mg/kg. In one embodiment, the dose is about 0.9 mg/kg. In one embodiment, the dose is about 1.3 mg/kg. In one embodiment, the dose is about 2.0 mg/kg. In certain embodiments, the dose is 0.65 mg/kg, 0.7 mg/kg, 0.75 mg/kg, 0.8 mg/kg, 0.85 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is 0.65 mg/kg. In one embodiment, the dose is 0.9 mg/kg. In one embodiment, the dose is 1.3 mg/kg. In one embodiment, the dose is 2.0 mg/kg. In some embodiments, the dose is 0.65 mg/kg and the anti-TF antibody-drug conjugate is tisotumab vedotin. In some embodiments, the dose is 0.9 mg/kg and the anti-TF antibody-drug conjugate is tisotumab vedotin. In some embodiments, the dose is 1.3 mg/kg and the anti-TF antibody-drug conjugate is tisotumab vedotin. In some embodiments, the dose is 2.0 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses or product for uses provided herein, an anti-TF antibody-drug conjugate or antigen-binding fragment thereof as described herein is administered to the subject once about every 1 to 4 weeks. In certain embodiments, an anti-TF antibody-drug conjugate or antigen-binding fragment thereof as described herein is administered once about every 1 week, once about every 2 weeks, once about every 3 weeks or once about every 4 weeks. In one embodiment, an anti-TF antibody-drug conjugate or antigen-binding fragment thereof as described herein is administered once about every 3 weeks. In one embodiment, an anti-TF antibody-drug conjugate or antigen-binding fragment thereof as described herein is administered once every 3 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., ±3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 1.3 mg/kg and is administered once every 3 weeks. In some embodiments, the dose is 1.3 mg/kg and is administered once every 3 weeks and the antibody-drug conjugate is tisotumab vedotin. In some embodiments, the dose is 1.3 mg/kg and is administered once every 3 weeks and the antibody-drug conjugate is tisotumab vedotin and the dose is decreased to 0.9 mg/kg if one or more adverse events occur. In some embodiments, the dose is about 0.9 mg/kg and is administered once about every week and the antibody-drug conjugate is tisotumab vedotin. In some embodiments, the dose is 0.9 mg/kg and is administered once every week and the antibody-drug conjugate is tisotumab vedotin. In some embodiments, the dose is 0.9 mg/kg and is administered on about days 1, 8, and 15 of about a 4-week cycle and the antibody-drug conjugate is tisotumab vedotin. In some embodiments, the dose is 0.9 mg/kg and is administered on days 1, 8, and 15 of a 4-week cycle and the antibody-drug conjugate is tisotumab vedotin. In some embodiments, the dose is 0.9 mg/kg and is administered on about days 1, 8, and 15 of about a 4-week cycle and the antibody drug conjugate is tisotumab vedotin and the dose is decreased to 0.65 mg/kg if one or more adverse events occur. In some embodiments, the dose is 0.9 mg/kg and is administered on days 1, 8, and 15 of a 4-week cycle and the antibody drug conjugate is tisotumab vedotin and the dose is decreased to 0.65 mg/kg if one or more adverse events occur. In some embodiments, the dose is about 0.65 mg/kg and is administered once about every week and the antibody-drug conjugate is tisotumab vedotin. In some embodiments, the dose is 0.65 mg/kg and is administered once every week and the antibody-drug conjugate is tisotumab vedotin. In some embodiments, the dose is 0.65 mg/kg and is administered on about days 1, 8, and 15 of about a 4-week cycle and the antibody drug conjugate is tisotumab vedotin. In some embodiments, the dose is 0.65 mg/kg and is administered on days 1, 8, and 15 of a 4-week cycle and the antibody drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses or product for uses provided herein, an anti-TF antibody-drug conjugate or antigen-binding fragment thereof as described herein is administered to the subject once about every 1 week for 3 consecutive weeks followed by about a 1 week rest period without any administration of the anti-TF antibody-drug conjugate or antigen-binding fragment thereof so that each cycle time is about 28 days including the resting period. In one embodiment of the methods or uses or product for uses provided herein, an anti-TF antibody-drug conjugate or antigen-binding fragment thereof as described herein is administered to the subject once every 1 week for 3 consecutive weeks followed by a 1 week rest period without any administration of the anti-TF antibody-drug conjugate or antigen-binding fragment thereof so that each cycle time is 28 days including the resting period. Hereby, a dosing regimen is provided where the subject to be treated is dosed with a single weekly dose for three consecutive weeks followed by a resting week. This treatment schedule may also be referred to as a “dose-dense schedule” herein and is the same as “the 4-week (28 days) cycle” and “3Q4W”. In one embodiment, an anti-TF antibody-drug conjugate or antigen-binding fragment thereof as described herein is administered to the subject on about days 1, 8, and 15 of about a 4-week cycle. In one embodiment, an anti-TF antibody-drug conjugate or antigen-binding fragment thereof as described herein is administered to the subject on days 1, 8, and 15 of a 4-week cycle. The present invention encompasses embodiments wherein the subject remains on the 3Q4W treatment cycle for at least 2, 3, 4, 5, 6, 7, 8, 9, 10, 11, 12 or more cycles. In another embodiment, the subject remains on the 3Q4W treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles wherein each cycle is 28 days as described above. In some embodiments, the subject remains on the 3Q4W treatment cycle for 12 cycles or more, such as 16 cycles or more, such as 24 cycles or more, such as 36 cycles or more. In some embodiments, the 3Q4W treatment cycle is administered for no more than 3, no more than 4, no more than 5, or no more than 6 four-week treatment cycles. The number of treatment cycles suitable for any specific subject or group of subjects may be determined by a person of skill in the art, typically a physician. In some embodiments, an anti-TF antibody-drug conjugate or antigen-binding fragment thereof as described herein is administered to the subject at a dose of about 0.9 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of about 0.9 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of about 0.9 mg/kg on about days 1, 8, and 15 of about a 4-week cycle. In some embodiments, an anti-TF antibody-drug conjugate or antigen-binding fragment thereof as described herein is administered to the subject at a dose of about 0.9 mg/kg on days 1, 8, and 15 of about a 4-week cycle. In some embodiments, an anti-TF antibody-drug conjugate or antigen-binding fragment thereof as described herein is administered to the subject at a dose of 0.9 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of 0.9 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of 0.9 mg/kg on about days 1, 8, and 15 of about a 4-week cycle. In some embodiments, an anti-TF antibody-drug conjugate or antigen-binding fragment thereof as described herein is administered to the subject at a dose of 0.9 mg/kg on days 1, 8, and 15 of about a 4-week cycle. In some embodiments, an anti-TF antibody-drug conjugate or antigen-binding fragment thereof as described herein is administered to the subject at a dose of about 0.65 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of about 0.65 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of about 0.65 mg/kg on about days 1, 8, and 15 of about a 4-week cycle. In some embodiments, an anti-TF antibody-drug conjugate or antigen-binding fragment thereof as described herein is administered to the subject at a dose of about 0.65 mg/kg on days 1, 8, and 15 of about a 4-week cycle. In some embodiments, an anti-TF antibody-drug conjugate or antigen-binding fragment thereof as described herein is administered to the subject at a dose of 0.65 mg/kg once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, an anti-TF antibody-drug conjugate or antigen-binding fragment thereof as described herein is administered to the subject at a dose of 0.65 mg/kg once every 1 week for 3 consecutive weeks followed by a 1 week rest period without any administration of the anti-TF antibody-drug conjugate or antigen-binding fragment thereof so that each cycle time is 28 days including the resting period. In some embodiments, an anti-TF antibody-drug conjugate or antigen-binding fragment thereof as described herein is administered to the subject at a dose of 0.65 mg/kg on about days 1, 8, and 15 of about a 4-week cycle. In some embodiments, an anti-TF antibody-drug conjugate or antigen-binding fragment thereof as described herein is administered to the subject at a dose of 0.65 mg/kg on days 1, 8, and 15 of about a 4-week cycle. In some embodiments, the dose is 0.9 mg/kg and is administered on about days 1, 8, and 15 of about a 4-week cycle and the antibody drug conjugate is tisotumab vedotin. In some embodiments, the dose is 0.9 mg/kg and is administered on days 1, 8, and 15 of a 4-week cycle and the antibody drug conjugate is tisotumab vedotin. In some embodiments, the dose is 0.9 mg/kg and is administered on about days 1, 8, and 15 of about a 4-week cycle and the antibody drug conjugate is tisotumab vedotin and the dose is decreased to 0.65 mg/kg if one or more adverse events occur. In some embodiments, the dose is 0.9 mg/kg and is administered on days 1, 8, and 15 of a 4-week cycle and the antibody drug conjugate is tisotumab vedotin and the dose is decreased to 0.65 mg/kg if one or more adverse events occur. In some embodiments, the dose is 0.65 mg/kg and is administered on about days 1, 8, and 15 of about a 4-week cycle and the antibody drug conjugate is tisotumab vedotin. In some embodiments, the dose is 0.65 mg/kg and is administered on days 1, 8, and 15 of a 4-week cycle and the antibody drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses or product for uses provided herein, an anti-TF antibody-drug conjugate or antigen-binding fragment thereof as described herein is administered to the subject at a flat dose ranging from about 50 mg to about 200 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 110 mg or a flat dose of about 120 mg or a flat dose of about 130 mg or a flat dose of about 140 mg or a flat dose of about 150 mg or a flat dose of about 160 mg or a flat dose of about 170 mg or a flat dose of about 180 mg or a flat dose of about 190 mg or a flat dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., f 3 days). In some embodiments, the flat dose is administered to the subject once every 3 weeks. In some embodiments, the flat dose is administered to the subject once every 3 weeks and the antibody-drug conjugate is tisotumab vedotin. In some embodiments, the flat does is administered to the subject once about every week (e.g., f 1 day). In some embodiments, the flat does is administered to the subject once every week. In some embodiments, the flat dose is administered to the subject once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, the flat dose is administered to the subject once every 1 week for 3 consecutive weeks followed by a 1 week rest period without any administration of the anti-TF antibody-drug conjugate or antigen-binding fragment thereof so that each cycle time is 28 days including the resting period. In some embodiments, the flat dose is administered to the subject on about days 1, 8, and 15 of about a 4-week cycle. In some embodiments, the flat dose is administered to the subject on days 1, 8, and 15 of a 4-week cycle. In some embodiments, the flat dose is administered to the subject on days 1, 8, and 15 of a 4-week cycle and the antibody-drug conjugate is tisotumab vedotin.

In one embodiment of the methods or uses or product for uses provided herein, an anti-TF antibody-drug conjugate or antigen-binding fragment thereof as described herein is administered to the subject at a flat dose ranging from 50 mg to 200 mg such as at a flat dose of 50 mg or a flat dose of 60 mg or a flat dose of 70 mg or a flat dose of 80 mg or a flat dose of 90 mg or a flat dose of 100 mg or a flat dose of 110 mg or a flat dose of 120 mg or a flat dose of 130 mg or a flat dose of 140 mg or a flat dose of 150 mg or a flat dose of 160 mg or a flat dose of 170 mg or a flat dose of 180 mg or a flat dose of 190 mg or a flat dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is tisotumab vedotin. In some embodiments, the flat does is administered to the subject once about every week (e.g., ±1 day). In some embodiments, the flat does is administered to the subject once every week. In some embodiments, the flat dose is administered to the subject once about every 1 week for 3 consecutive weeks followed by about a 1 week rest period without any administration of the anti-TF antibody-drug conjugate or antigen-binding fragment thereof so that each cycle time is about 28 days including the resting period. In some embodiments, the flat dose is administered to the subject once every 1 week for 3 consecutive weeks followed by a 1 week rest period without any administration of the anti-TF antibody-drug conjugate or antigen-binding fragment thereof so that each cycle time is 28 days including the resting period. In some embodiments, the flat dose is administered to the subject on about days 1, 8, and 15 of about a 4-week cycle. In some embodiments, the flat dose is administered to the subject on days 1, 8, and 15 of a 4-week cycle. In some embodiments, the flat dose is administered to the subject on days 1, 8, and 15 of a 4-week cycle and the antibody-drug conjugate is tisotumab vedotin.

In some embodiments, a method of treatment or use or product for use described herein further comprises the administration of one or more additional therapeutic agents. In some embodiments, the one or more additional therapeutic agents are administered simultaneously with an anti-TF antibody-drug conjugate or antigen-binding fragment thereof as described herein, such as tisotumab vedotin. In some embodiments, the one or more additional therapeutic agents and an anti-TF antibody-drug conjugate or antigen-binding fragment thereof as described herein are administered sequentially. In some embodiments, simultaneous means that the anti-TF antibody-drug conjugate and the one or more additional therapeutic agents are administered to the subject less than one hour apart, such as less than about 30 minutes apart, less than about 15 minutes apart, less than about 10 minutes apart or less than about 5 minutes apart. In some embodiments, sequential administration means that the anti-TF antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

C. Treatment Outcome

In one aspect, a method of treating ovarian cancer, peritoneal cancer, or fallopian tube cancer with an anti-TF antibody-drug conjugate or antigen-binding fragment thereof as described herein, such as e.g., tisotumab vedotin, results in an improvement in one or more therapeutic effects in the subject after administration of the antibody-drug conjugate relative to a baseline. In some embodiments, the one or more therapeutic effects is the size of the tumor derived from the cancer, the objective response rate, the duration of response, the time to response, progression free survival, overall survival, CA-125 level, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression. In one embodiment, the one or more therapeutic effects CA-125 level.

In one embodiment of the methods or uses or product for uses provided herein, response to treatment with an anti-TF antibody-drug conjugate or antigen-binding fragment thereof as described herein, such as e.g., tisotumab vedotin, may include the following criteria (RECIST Criteria 1.1):

| | Category | Criteria |
|---|---|---|
| Based on | Complete | Disappearance of all target lesions. |
| target | Response | Any pathological lymph nodes |
| lesions | (CR) | must have reduction in short axis |
| | | to <10 mm. |
| | Partial | ≥30% decrease in the sum of the |
| | Response | longest diameter (LD) of target |
| | (PR) | lesions, taking as reference the |
| | | baseline sum of LDs. |
| | Stable | Neither sufficient shrinkage to |
| | Disease | qualify for PR nor sufficient increase |
| | (SD) | to qualify for PD, taking as reference |
| | | the smallest sum of LDs while in trial. |
| | Progressive | ≥20% (and ≥5 mm) increase in the |
| | Disease | sum of the LDs of target lesions, taking |
| | (PD) | as reference the smallest sum of the |
| | | target LDs recorded while in trial or the |
| | | appearance of one or more new lesions. |
| Based on | CR | Disappearance of all non-target lesions |
| non-target | | and normalization of tumor marker level. |
| lesions | | All lymph nodes must be non- |
| | | pathological in size (<10 mm short axis). |
| | SD | Persistence of one or more non-target |
| | | lesion(s) or/and maintenance of tumor |
| | | marker level above the normal limits. |
| | PD | Appearance of one or more new lesions |
| | | and/or unequivocal progression of |
| | | existing non-target lesions. |

In one embodiment of the methods or uses or product for uses provided herein, the effectiveness of treatment with an anti-TF antibody-drug conjugate or antigen-binding fragment thereof described herein, such as e.g., tisotumab vedotin, is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-TF antibody-drug conjugate or antigen-binding fragment thereof described herein, such as e.g., tisotumab vedotin, is assessed by measuring the size of a tumor derived from the ovarian cancer, peritoneal cancer, or fallopian tube cancer. In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound.

In one embodiment of the methods or uses or product for uses provided described herein, response to treatment with an antibody-drug conjugate or antigen-binding fragment thereof described herein, such as e.g., tisotumab vedotin, promotes regression of a tumor derived from the ovarian cancer, peritoneal cancer, or fallopian tube cancer. In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In one embodiment of the methods or uses or product for uses described herein, response to treatment with an anti-TF antibody-drug conjugate or antigen-binding fragment thereof described herein, such as e.g., tisotumab vedotin, is assessed by measuring the time of progression free survival after administration of the anti-TF antibody-drug conjugate. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the subject exhibits progression-free survival of at least about 6 months after administration of the anti-TF antibody-drug conjugate. In some embodiments, the subject exhibits progression-free survival of at least about one year after administration of the anti-TF antibody-drug conjugate. In some embodiments, the subject exhibits progression-free survival of at least about two years after administration of the anti-TF antibody-drug conjugate. In some embodiments, the subject exhibits progression-free survival of at least about three years after administration of the anti-TF antibody-drug conjugate. In some embodiments, the subject exhibits progression-free survival of at least about four years after administration of the anti-TF antibody-drug conjugate. In some embodiments, the subject exhibits progression-free survival of at least about five years after administration of the anti-TF antibody-drug conjugate. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the subject exhibits progression-free survival of at least 6 months after administration of the anti-TF antibody-drug conjugate. In some embodiments, the subject exhibits progression-free survival of at least one year after administration of the anti-TF antibody-drug conjugate. In some embodiments, the subject exhibits progression-free survival of at least two years after administration of the anti-TF antibody-drug conjugate. In some embodiments, the subject exhibits progression-free survival of at least three years after administration of the anti-TF antibody-drug conjugate. In some embodiments, the subject exhibits progression-free survival of at least four years after administration of the anti-TF antibody-drug conjugate. In some embodiments, the subject exhibits progression-free survival of at least five years after administration of the anti-TF antibody-drug conjugate.

In one embodiment of the methods or uses or product for uses described herein, response to treatment with an anti-TF antibody-drug conjugate or antigen-binding fragment thereof described herein, such as e.g., tisotumab vedotin, is assessed by measuring the time of overall survival after administration of the anti-TF antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the subject exhibits overall survival of at least about 6 months after administration of the anti-TF antibody-drug conjugate. In some embodiments, the subject exhibits overall survival of at least about one year after administration of the anti-TF antibody-drug conjugate. In some embodiments, the subject exhibits overall survival of at least about two years after administration of the anti-TF antibody-drug conjugate. In some embodiments, the subject exhibits overall survival of at least about three years after administration of the anti-TF antibody-drug conjugate. In some embodiments, the subject exhibits overall survival of at least about four years after administration of the anti-TF antibody-drug conjugate. In some embodiments, the subject exhibits overall survival of at least about five years after administration of the anti-TF antibody-drug conjugate. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the subject exhibits overall survival of at least 6 months after administration of the anti-TF antibody-drug conjugate. In some embodiments, the subject exhibits overall survival of at least one year after administration of the anti-TF antibody-drug conjugate. In some embodiments, the subject exhibits overall survival of at least two years after administration of the anti-TF antibody-drug conjugate. In some embodiments, the subject exhibits overall survival of at least three years after administration of the anti-TF antibody-drug conjugate. In some embodiments, the subject exhibits overall survival of at least four years after administration of the anti-TF antibody-drug conjugate. In some embodiments, the subject exhibits overall survival of at least five years after administration of the anti-TF antibody-drug conjugate.

In one embodiment of the methods or uses or product for uses described herein, response to treatment with an anti-TF antibody-drug conjugate or antigen-binding fragment thereof described herein, such as e.g., tisotumab vedotin, is assessed by measuring the duration of response to the anti-TF antibody-drug conjugate after administration of the anti-TF antibody-drug conjugate. In some embodiments, the duration of response to the anti-TF antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate. In some embodiments, the duration of response to the anti-TF antibody-drug conjugate is at least about 6 months after administration of the antibody-drug conjugate. In some embodiments, the duration of response to the anti-TF antibody-drug conjugate is at least about one year after administration of the antibody-drug conjugate. In some embodiments, the duration of response to the anti-TF antibody-drug conjugate is at least about two years after administration of the antibody-drug conjugate. In some embodiments, the duration of response to the anti-TF antibody-drug conjugate is at least about three years after administration of the antibody-drug conjugate. In some embodiments, the duration of response to the anti-TF antibody-drug conjugate is at least about four years after administration of the antibody-drug conjugate. In some embodiments, the duration of response to the anti-TF antibody-drug conjugate is at least about five years after administration of the antibody-drug conjugate. In some embodiments, the duration of response to the anti-TF antibody-drug conjugate is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate. In some embodiments, the duration of response to the anti-TF antibody-drug conjugate is at least 6 months after administration of the antibody-drug conjugate. In some embodiments, the duration of response to the anti-TF antibody-drug conjugate is at least one year after administration of the antibody-drug conjugate. In some embodiments, the duration of response to the anti-TF antibody-drug conjugate is at least two years after administration of the antibody-drug conjugate. In some embodiments, the duration of response to the anti-TF antibody-drug conjugate is at least three years after administration of the antibody-drug conjugate. In some embodiments, the duration of response to the anti-TF antibody-drug conjugate is at least four years after administration of the antibody-drug conjugate. In some embodiments, the duration of response to the anti-TF antibody-drug conjugate is at least five years after administration of the antibody-drug conjugate.

In one embodiment of the methods or uses or product for uses described herein, response to treatment with an anti-TF antibody-drug conjugate or antigen-binding fragment thereof described herein, such as e.g., tisotumab vedotin, is assessed by measuring the cancer antigen-125 (CA-125) level in a blood sample from the subject. In some embodiments, the CA-125 response rate is according to Gynecologic Cancer Intergroup (GCIG) criteria. See Rustin et al., 2011, Int. J. Gynecol. Cancer 21(2)413-23. In some embodiments, the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample from the subject obtained before administration of the antibody-drug conjugate.

D. Adverse Events

In one aspect, a method of treating ovarian cancer with an anti-TF antibody-drug conjugates or antigen-binding fragments thereof described herein, such as e.g., tisotumab vedotin, results in the subject developing one or more adverse events. In some embodiments, the subject is administered an additional therapeutic agent to eliminate or reduce the severity of the adverse event. In some embodiments, the one or more adverse events the subject develops is anaphylaxis, anemia, abdominal pain, hypokalemia, hyponatremia, severe hypersensitivity, epistaxis, an infusion-related reaction, fatigue, nausea, alopecia, conjunctivitis, symblepharon, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and the additional therapeutic agent is a preservative-free lubricating eye drop, an ocular vasoconstrictor, an antibiotic, a steroid eye drop, or any combination thereof. In some embodiments, the one or more adverse events is keratitis and the additional therapeutic agent is a preservative-free lubricating eye drop, an ocular vasoconstrictor, an antibiotic, a steroid eye drop, or any combination thereof. In some of any of the embodiments herein, the subject is administered a treatment with the additional therapeutic agent to eliminate or reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

In one aspect, the subject treated with an anti-TF antibody-drug conjugates or antigen-binding fragments thereof described herein, such as e.g., tisotumab vedotin, is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, an antibiotic, a steroid eye drop, or any combination thereof. In some embodiments, the one or more adverse events is conjunctivitis and the additional therapeutic agent is a preservative-free lubricating eye drop, an ocular vasoconstrictor, an antibiotic, a steroid eye drop, or any combination thereof. In some embodiments, the one or more adverse events is keratitis and the additional therapeutic agent is a preservative-free lubricating eye drop, an ocular vasoconstrictor, an antibiotic, a steroid eye drop, or any combination thereof. In some of any of the embodiments herein, the subject is administered a treatment with the additional therapeutic agent to prevent the development of the adverse event or to reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

### IV. Compositions

In some aspects, also provided herein are compositions (e.g., pharmaceutical compositions and therapeutic formulations) comprising any of the anti-TF antibody-drug conjugates or antigen-binding fragments thereof described herein, such as e.g., tisotumab vedotin.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wilkins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, omithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaceharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating ovarian cancer as described herein. In some aspects, provided herein are compositions comprising a population of antibody-drug conjugates, wherein the antibody-drug conjugates comprise a linker attached to MMAE, wherein is the antibody-drug conjugate has the following structure:

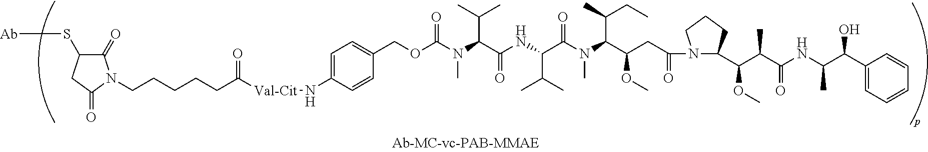

wherein p denotes a number from 1 to 8, e.g., 1, 2, 3, 4, 5, 6, 7 or 8. S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an anti-TF antibody-drug conjugate as described herein, such as e.g., tisotumab vedotin, is coadministered with one or more additional therapeutic agents. In some embodiments the coadministration is simultaneous or sequential. In some embodiments, the anti-TF antibody-drug conjugate as described herein is administered simultaneously with the one or more additional therapeutic agents. In some embodiments, simultaneous means that the anti-TF antibody-drug conjugate and the one or more additional therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate and the one or more additional therapeutic agents are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the one or more additional therapeutic agents. In some embodiments, sequential administration means that the anti-TF antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-TF antibody-drug conjugate as described herein, such as e.g., tisotumab vedotin, is coadministered with one or more therapeutic agents to eliminate or reduce the severity of one or more adverse events. In some embodiments the coadministration is simultaneous or sequential. In some embodiments, the anti-TF antibody-drug conjugate is administered simultaneously with the one or more therapeutic agents to eliminate or reduce the severity of one or more adverse events. In some embodiments, simultaneous means that the anti-TF antibody-drug conjugate and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the one or more therapeutic agents to eliminate or reduce the severity of one or more adverse events. In some embodiments, sequential administration means that the anti-TF antibody-drug conjugate and the one or more therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-TF antibody-drug conjugate is administered prior to the one or more therapeutic agents to eliminate or reduce the severity of one or more adverse events. In some embodiments, the one or more therapeutic agents to eliminate or reduce the severity of one or more adverse events is administered prior to the anti-TF antibody-drug conjugate.

### V. Articles of Manufacture and Kits

In another aspect, an article of manufacture or kit is provided which comprises an anti-TF antibody-drug conjugate described herein, such as e.g., tisotumab vedotin. The article of manufacture or kit may further comprise instructions for use of the anti-TF antibody-drug conjugate in the methods of the invention. Thus, in certain embodiments, the article of manufacture or kit comprises instructions for the use of an anti-TF antibody-drug conjugate in methods for treating ovarian cancer in a subject comprising administering to the subject an effective amount of an anti-TF antibody-drug conjugate. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating ovarian cancer as described herein in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

The article of manufacture or kit herein optionally further comprises a container comprising a second medicament, wherein the second medicament is for eliminating or reducing the severity of one or more adverse events, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

In some embodiments, the anti-TF antibody-drug conjugate is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VI. Exemplary Embodiments

Among the embodiments provided herein are:

1. A method of treating ovarian cancer, peritoneal cancer or fallopian tube cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.65 mg/kg to about 2.1 mg/kg.
2. The method of embodiment 1, wherein the dose is about 2.0 mg/kg.
3. The method of embodiment 1, wherein the dose is 2.0 mg/kg.
4. The method of any one of embodiments 1-3, wherein the antibody-drug conjugate is administered once about every 3 weeks.
5. The method of any one of embodiments 1-3, wherein the antibody-drug conjugate is administered once every 3 weeks.
6. The method of embodiment 1, wherein the dose is about 0.65 mg/kg.
7. The method of embodiment 1, wherein the dose is 0.65 mg/kg.
8. The method of embodiment 1, wherein the dose is about 0.9 mg/kg.
9. The method of embodiment 1, wherein the dose is 0.9 mg/kg.
10. The method of any one of embodiments 1 or 6-9, wherein the antibody-drug conjugate is administered once about every week.
11. The method of any one of embodiments 1 or 6-9, wherein the antibody-drug conjugate is administered once every week.
12. The method of any one of embodiments 1 or 6-9, wherein the antibody-drug conjugate is administered once about every 1 week for 3 consecutive weeks followed by about a 1 week resting period during which the antibody-drug conjugate is not administered.
13. The method of any one of embodiments 1 or 6-9, wherein the antibody-drug conjugate is administered once every 1 week for three consecutive weeks followed by a one week resting period during which the antibody-drug conjugate is not administered.
14. The method of any one of embodiments 1 or 6-9, wherein the antibody-drug conjugate is administered on about days 1, 8, and 15 of about a 4-week cycle.
15. The method of any one of embodiments 1 or 6-9, wherein the antibody-drug conjugate is administered on days 1, 8, and 15 of a 4-week cycle.
16. The method of any one of embodiments 1-15, wherein the subject has been previously treated with one or more therapeutic agents and did not respond to the treatment, wherein the one or more therapeutic agents is not the antibody-drug conjugate.
17. The method of any one of embodiments 1-15, wherein the subject has been previously treated with one or more therapeutic agents and relapsed after the treatment, wherein the one or more therapeutic agents is not the antibody-drug conjugate.
18. The method of any one of embodiments 1-15, wherein the subject has been previously treated with one or more therapeutic agents and has experienced disease progression during treatment, wherein the one or more therapeutic agents is not the antibody-drug conjugate.
19. The method of any one of embodiments 1-18, wherein the subject has been previously treated with a platinum-based therapy.
20. The method of embodiment 19, wherein the cancer is platinum-resistant.
21. The method of embodiment 20, wherein the subject experienced disease progression or relapsed 2 or more months after treatment with the platinum-based therapy.
22. The method of embodiment 20, wherein the subject experienced disease progression or relapsed within 6 months after treatment with the platinum-based therapy.
23. The method of embodiment 20, wherein the subject experienced disease progression or relapsed between 2 months and 6 months after treatment with the platinum-based therapy.
24. The method of any one of embodiments 19-23, wherein cancer is not platinum-refractory.
25. The method of any one of embodiments 19-24, wherein the subject did not experience disease progression or relapse within 2 months after treatment with the platinum-based therapy.
26. The method of any one of embodiments 1-25, wherein the subject has been previously treated with a VEGF antagonist.
27. The method of embodiment 26, wherein the VEGF antagonist is an anti-VEGF antibody.
28. The method of embodiment 27, wherein the anti-VEGF antibody is bevacizumab.
29. The method of any one of embodiments 1-28, wherein the subject received prior systemic therapy and experienced disease progression on or after the systemic therapy.
30. The method of any one of embodiments 1-29, wherein the subject received 1, 2, 3, 4 or 5 rounds of prior systemic therapy.
31. The method of embodiment 30, wherein the prior systemic therapy is a chemotherapy regimen and wherein poly ADP ribose polymerase (PARP) inhibitors are not chemotherapy.
32. The method of any one of embodiments 1-31, wherein the cancer is ovarian cancer.
33. The method of embodiment 32, wherein the ovarian cancer is epithelial ovarian cancer.
34. The method of any one of embodiments 1-31, wherein the cancer is peritoneal cancer.
35. The method of embodiment 34, wherein the peritoneal cancer is primary peritoneal cancer.
36. The method of any one of embodiments 1-31, wherein the cancer is fallopian tube cancer.
37. The method of any one of embodiments 1-36, wherein the cancer is an advanced stage cancer.
38. The method of embodiment 37, wherein the advanced stage cancer is a stage 3 or stage 4 cancer.
39. The method of embodiment 37 or embodiment 38, wherein the advanced stage cancer is metastatic cancer.
40. The method of any one of embodiments 1-39, wherein the cancer is recurrent cancer.
41. The method of any one of embodiments 1-40, wherein the monomethyl auristatin is monomethyl auristatin E (MMAE).
42. The method of any one of embodiments 1-41, wherein the anti-TF antibody or antigen-binding fragment thereof of the antibody-drug conjugate is a monoclonal antibody or a monoclonal antigen-binding fragment thereof.
43. The method of any one of embodiments 1-42, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

44. The method of any one of embodiments 1-43, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
45. The method of any one of embodiments 1-44, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
46. The method of any one of embodiments 1-45, wherein the anti-TF antibody of the antibody-drug conjugate is tisotumab.
47. The method of any one of embodiments 1-46, wherein the antibody-drug conjugate further comprises a linker between the anti-TF antibody or antigen-binding fragment thereof and the monomethyl auristatin.
48. The method of embodiment 47, wherein the linker is a cleavable peptide linker.
49. The method of embodiment 48, wherein the cleavable peptide linker has a formula: -MC-vc-PAB-, wherein:

a) MC is:

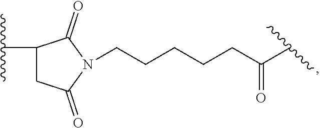 [SMILES: *C(=O)CCCCCN1C(=O)CC(*)C1=O]

b) vc is the dipeptide valine-citrulline, and

c) PAB is:

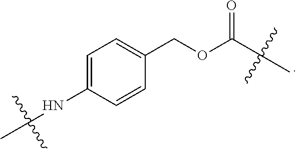 [SMILES: *Nc1ccc(COC(*)=O)cc1]

50. The method of any one of embodiments 47-49, wherein the linker is attached to sulphydryl residues of the anti-TF antibody obtained by partial reduction or full reduction of the anti-TF antibody or antigen-binding fragment thereof.
51. The method of embodiment 50, wherein the linker is attached to monomethyl auristatin E (MMAE), wherein the antibody-drug conjugate has the following structure:

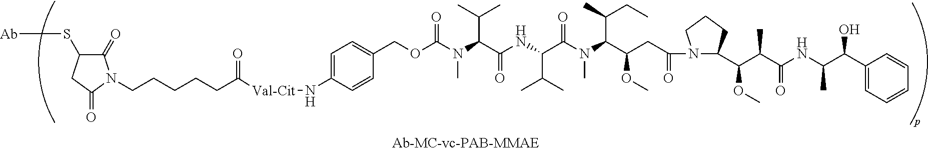

wherein p denotes a number from 1 to 8, S represents a sulphydryl residue of the anti-TF antibody, and Ab designates the anti-TF antibody or antigen-binding fragment thereof.
52. The method of embodiment 51, wherein the average value of p in a population of the antibody-drug conjugates is about 4.
53. The method of any one of embodiments 1-52, wherein the antibody-drug conjugate is tisotumab vedotin.
54. The method of any one of embodiments 1-53, wherein the route of administration for the antibody-drug conjugate is intravenous.
55. The method of any one of embodiments 1-54, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
56. The method of any one of embodiments 1-55, wherein one or more therapeutic effects in the subject is improved after administration of the antibody-drug conjugate relative to a baseline.
57. The method of embodiment 56, wherein the one or more therapeutic effects is selected from the group consisting of: size of a tumor derived from the cancer, objective response rate, duration of response, time to response, progression free survival, overall survival and CA-125 level.
58. The method of any one of embodiments 1-57, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
59. The method of any one of embodiments 1-58, wherein the objective response rate is at least about 20%, at least about 25%, at least about 30%, at least about 35%, at least about 40%, at least about 45%, at least about 50%, at least about 60%, at least about 70%, or at least about 80%.
60. The method of any one of embodiments 1-59, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
61. The method of any one of embodiments 1-60, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
62. The method of any one of embodiments 1-61, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
63. The method of any one of embodiments 1-62, wherein the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample obtained from the subject before administration of the antibody-drug conjugate.
64. The method of any one of embodiments 1-63, wherein the subject has one or more adverse events and is further administered an additional therapeutic agent to eliminate or reduce the severity of the one or more adverse events.
65. The method of any one of embodiments 1-64, wherein the subject is at risk of developing one or more adverse events and is further administered an additional therapeutic agent to prevent or reduce the severity of the one or more adverse events.
66. The method of embodiment 64 or embodiment 65, wherein the one or more adverse events is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, or general physical health deterioration.
67. The method of any one of embodiments 64-66, wherein the one or more adverse events is a grade 3 or greater adverse event.
68. The method of nay one of embodiments 64-66, wherein the one or more adverse events is a serious adverse event.
69. The method of any one of embodiments 64-68, wherein the one or more adverse events is conjunctivitis and/or keratitis and the additional agent is a preservative-free lubricating eye drop, an ocular vasoconstrictor and/or a steroid eye drop.
70. The method of any one of embodiments 1-69, wherein the antibody-drug conjugate is administered as a monotherapy.
71. The method of any one of embodiments 1-70, wherein the subject is a human.
72. The method of any one of embodiments 1-71, wherein the antibody-drug conjugate is in a pharmaceutical composition comprising the antibody-drug conjugate and a pharmaceutical acceptable carrier.
73. A kit comprising:

(a) a dosage ranging from about 0.65 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and

(b) instructions for using the antibody drug conjugate according to the method of any one of embodiments 1-72.

74. An antibody-drug conjugate that binds to TF for use in the treatment of ovarian cancer, peritoneal cancer or fallopian tube cancer in a subject, wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.65 mg/kg to about 2.1 mg/kg.
75. The antibody-drug conjugate for use of embodiment 74, wherein the dose is about 2.0 mg/kg.
76. The antibody-drug conjugate for use of embodiment 74, wherein the dose is 2.0 mg/kg.
77. The antibody-drug conjugate for use of any one of embodiments 74-76, wherein the antibody-drug conjugate is administered once about every 3 weeks.
78. The antibody-drug conjugate for use of any one of embodiments 74-76, wherein the antibody-drug conjugate is administered once every 3 weeks.
79. The antibody-drug conjugate for use of embodiment 74, wherein the dose is about 0.65 mg/kg.
80. The antibody-drug conjugate for use of embodiment 74, wherein the dose is 0.65 mg/kg.
81. The antibody-drug conjugate for use of embodiment 74, wherein the dose is about 0.9 mg/kg.
82. The antibody-drug conjugate for use of embodiment 74, wherein the dose is 0.9 mg/kg.
83. The antibody-drug conjugate for use of any one of embodiments 74 or 79-82, wherein the antibody-drug conjugate is administered once about every week.
84. The antibody-drug conjugate for use of any one of embodiments 74 or 79-82, wherein the antibody-drug conjugate is administered once every week.
85. The antibody-drug conjugate for use of any one of embodiments 74 or 79-82, wherein the antibody-drug conjugate is administered once about every 1 week for 3 consecutive weeks followed by about a 1 week resting period during which the antibody-drug conjugate is not administered.
86. The antibody-drug conjugate for use of any one of embodiments 74 or 79-82, wherein the antibody-drug conjugate is administered once every 1 week for three consecutive weeks followed by a one week resting period during which the antibody-drug conjugate is not administered.
87. The antibody-drug conjugate for use of any one of embodiments 74 or 79-82, wherein the antibody-drug conjugate is administered on about days 1, 8, and 15 of about a 4-week cycle.
88. The antibody-drug conjugate for use of any one of embodiments 74 or 79-82, wherein the antibody-drug conjugate is administered on days 1, 8, and 15 of a 4-week cycle.
89. The antibody-drug conjugate for use of any one of embodiments 74-88, wherein the subject has been previously treated with one or more therapeutic agents and did not respond to the treatment, wherein the one or more therapeutic agents is not the antibody-drug conjugate.
90. The antibody-drug conjugate for use of any one of embodiments 74-88, wherein the subject has been previously treated with one or more therapeutic agents and relapsed after the treatment, wherein the one or more therapeutic agents is not the antibody-drug conjugate.
91. The antibody-drug conjugate for use of any one of embodiments 74-88, wherein the subject has been previously treated with one or more therapeutic agents and has experienced disease progression during treatment, wherein the one or more therapeutic agents is not the antibody-drug conjugate.
92. The antibody-drug conjugate for use of any one of embodiments 74-91, wherein the subject has been previously treated with a platinum-based therapy.
93. The antibody-drug conjugate for use of embodiment 92, wherein the cancer is platinum-resistant.
94. The antibody-drug conjugate for use of embodiment 93, wherein the subject experienced disease progression or relapsed 2 or more months after treatment with the platinum-based therapy.
95. The antibody-drug conjugate for use of embodiment 93, wherein the subject experienced disease progression or relapsed within 6 months after treatment with the platinum-based therapy.
96. The antibody-drug conjugate for use of embodiment 93, wherein the subject experienced disease progression or relapsed between 2 months and 6 months after treatment with the platinum-based therapy.
97. The antibody-drug conjugate for use of any one of embodiments 92-96, wherein cancer is not platinum-refractory.
98. The antibody-drug conjugate for use of any one of embodiments 92-97, wherein the subject did not experience disease progression or relapse within 2 months after treatment with the platinum-based therapy.
99. The antibody-drug conjugate for use of any one of embodiments 74-98, wherein the subject has been previously treated with a VEGF antagonist.
100. The antibody-drug conjugate for use of embodiment 99, wherein the VEGF antagonist is an anti-VEGF antibody.
101. The antibody-drug conjugate for use of embodiment 100, wherein the anti-VEGF antibody is bevacizumab.
102. The antibody-drug conjugate for use of any one of embodiments 74-101, wherein the subject received prior systemic therapy and experienced disease progression on or after the systemic therapy.
103. The antibody-drug conjugate for use of any one of embodiments 74-102, wherein the subject received 1, 2, 3, 4 or 5 rounds of prior systemic therapy.
104. The antibody-drug conjugate for use of embodiment 103, wherein the prior systemic therapy is a chemotherapy regimen and wherein poly ADP ribose polymerase (PARP) inhibitors are not chemotherapy.
105. The antibody-drug conjugate for use of any one of embodiments 74-104, wherein the cancer is ovarian cancer.
106. The antibody-drug conjugate for use of embodiment 105, wherein the ovarian cancer is epithelial ovarian cancer.
107. The antibody-drug conjugate for use of any one of embodiments 74-104, wherein the cancer is peritoneal cancer.
108. The antibody-drug conjugate for use of embodiment 107, wherein the peritoneal cancer is primary peritoneal cancer.
109. The antibody-drug conjugate for use of any one of embodiments 74-104, wherein the cancer is fallopian tube cancer.
110. The antibody-drug conjugate for use of any one of embodiments 74-109, wherein the cancer is an advanced stage cancer.
111. The antibody-drug conjugate for use of embodiment 110, wherein the advanced stage cancer is a stage 3 or stage 4 cancer.
112. The antibody-drug conjugate for use of embodiment 110 or embodiment 111, wherein the advanced stage cancer is metastatic cancer.
113. The antibody-drug conjugate for use of any one of embodiments 74-112, wherein the cancer is recurrent cancer.
114. The antibody-drug conjugate for use of any one of embodiments 74-113, wherein the monomethyl auristatin is monomethyl auristatin E (MMAE).
115. The antibody-drug conjugate for use of any one of embodiments 74-114, wherein the anti-TF antibody or antigen-binding fragment thereof of the antibody-dug conjugate is a monoclonal antibody or a monoclonal antigen-binding fragment thereof.
116. The antibody-drug conjugate for use of any one of embodiments 74-115, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

117. The antibody-drug conjugate for use of any one of embodiments 74-116, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
118. The antibody-drug conjugate for use of any one of embodiments 74-117, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
119. The antibody-drug conjugate for use of any one of embodiments 74-118, wherein the anti-TF antibody of the antibody-drug conjugate is tisotumab.
120. The antibody-drug conjugate for use of any one of embodiments 74-119, wherein the antibody-drug conjugate further comprises a linker between the anti-TF antibody or antigen-binding fragment thereof and the monomethyl auristatin.
121. The antibody-drug conjugate for use of embodiment 120, wherein the linker is a cleavable peptide linker.
122. The antibody-drug conjugate for use of embodiment 121, wherein the cleavable peptide linker has a formula: -MC-vc-PAB-, wherein:

a) MC is:

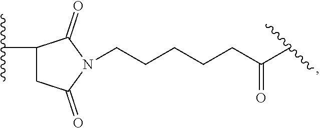 [SMILES: *C(=O)CCCCCN1C(=O)CC(*)C1=O]

b) vc is the dipeptide valine-citrulline, and

c) PAB is:

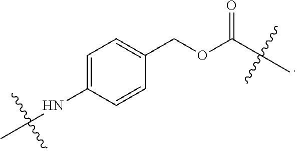 [SMILES: *Nc1ccc(COC(*)=O)cc1]

123. The antibody-drug conjugate for use of any one of embodiments 120-122, wherein the linker is attached to sulphydryl residues of the anti-TF antibody obtained by partial reduction or full reduction of the anti-TF antibody or antigen-binding fragment thereof.
124. The antibody-drug conjugate for use of embodiment 123, wherein the linker is attached to monomethyl auristatin E (MMAE), wherein the antibody-drug conjugate has the following structure:

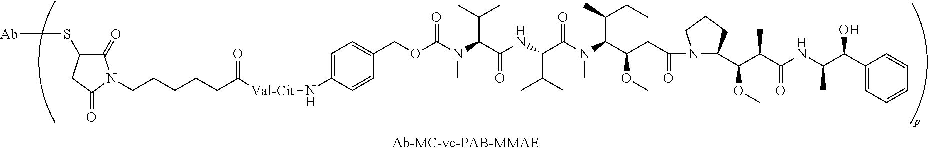

wherein p denotes a number from 1 to 8, S represents a sulphydryl residue of the anti-TF antibody, and Ab designates the anti-TF antibody or antigen-binding fragment thereof.
125. The antibody-drug conjugate for use of embodiment 124, wherein the average value of p in a population of the antibody-drug conjugates is about 4.
126. The antibody-drug conjugate for use of any one of embodiments 74-125, wherein the antibody-drug conjugate is tisotumab vedotin.
127. The antibody-drug conjugate for use of any one of embodiments 74-126, wherein the route of administration for the antibody-drug conjugate is intravenous.
128. The antibody-drug conjugate for use of any one of embodiments 74-127, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
129. The antibody-drug conjugate for use of any one of embodiments 74-128, wherein one or more therapeutic effects in the subject is improved after administration of the antibody-drug conjugate relative to a baseline.
130. The antibody-drug conjugate for use of embodiment 129, wherein the one or more therapeutic effects is selected from the group consisting of: size of a tumor derived from the cancer, objective response rate, duration of response, time to response, progression free survival, overall survival and CA-125 level.
131. The antibody-drug conjugate for use of any one of embodiments 74-130, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
132. The antibody-drug conjugate for use of any one of embodiments 74-131, wherein the objective response rate is at least about 20%, at least about 25%, at least about 30%, at least about 35%, at least about 40%, at least about 45%, at least about 50%, at least about 60%, at least about 70%, or at least about 80%.
133. The antibody-drug conjugate for use of any one of embodiments 74-132, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
134. The antibody-drug conjugate for use of any one of embodiments 74-133, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
135. The antibody-drug conjugate for use of any one of embodiments 74-134, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
136. The antibody-drug conjugate for use of any one of embodiments 74-135, wherein the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample obtained from the subject before administration of the antibody-drug conjugate.
137. The antibody-drug conjugate for use of any one of embodiments 74-136, wherein the subject has one or more adverse events and is further administered an additional therapeutic agent to eliminate or reduce the severity of the one or more adverse events.
138. The antibody-drug conjugate for use of any one of embodiments 74-137, wherein the subject is at risk of developing one or more adverse events and is further administered an additional therapeutic agent to prevent or reduce the severity of the one or more adverse events.
139. The antibody-drug conjugate for use of embodiment 137 or embodiment 138, wherein the one or more adverse events is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, or general physical health deterioration.
140. The antibody-drug conjugate for use of any one of embodiments 137-139, wherein the one or more adverse events is a grade 3 or greater adverse event.
141. The antibody-drug conjugate for use of nay one of embodiments 137-139, wherein the one or more adverse events is a serious adverse event.
142. The antibody-drug conjugate for use of any one of embodiments 137-141, wherein the one or more adverse events is conjunctivitis and/or keratitis and the additional agent is a preservative-free lubricating eye drop, an ocular vasoconstrictor and/or a steroid eye drop.
143. The antibody-drug conjugate for use of any one of embodiments 74-142, wherein the antibody-drug conjugate is administered as a monotherapy.
144. The antibody-drug conjugate for use of any one of embodiments 74-143, wherein the subject is a human.
145. The antibody-drug conjugate for use of any one of embodiments 74-144, wherein the antibody-drug conjugate is in a pharmaceutical composition comprising the antibody-drug conjugate and a pharmaceutical acceptable carrier.
146. Use of an antibody-drug conjugate that binds to tissue factor (TF) for the manufacture of a medicament for treating ovarian cancer, peritoneal cancer or fallopian tube cancer in a subject, wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.65 mg/kg to about 2.1 mg/kg.
147. The use of embodiment 146, wherein the dose is about 2.0 mg/kg.
148. The use of embodiment 146, wherein the dose is 2.0 mg/kg.
149. The use of any one of embodiments 146-148, wherein the antibody-drug conjugate is administered once about every 3 weeks.
150. The use of any one of embodiments 146-148, wherein the antibody-drug conjugate is administered once every 3 weeks.
151. The use of embodiment 146, wherein the dose is about 0.65 mg/kg.
152. The use of embodiment 146, wherein the dose is 0.65 mg/kg.
153. The use of embodiment 146, wherein the dose is about 0.9 mg/kg.
154. The use of embodiment 146, wherein the dose is 0.9 mg/kg.
155. The use of any one of embodiments 146 or 151-154, wherein the antibody-drug conjugate is administered once about every week.
156. The use of any one of embodiments 146 or 151-154, wherein the antibody-drug conjugate is administered once every week.
157. The use of any one of embodiments 146 or 151-154, wherein the antibody-drug conjugate is administered once about every 1 week for 3 consecutive weeks followed by about a 1 week resting period during which the antibody-drug conjugate is not administered.
158. The use of any one of embodiments 146 or 151-154, wherein the antibody-drug conjugate is administered once every 1 week for three consecutive weeks followed by a one week resting period during which the antibody-drug conjugate is not administered.
159. The use of any one of embodiments 146 or 151-154, wherein the antibody-drug conjugate is administered on about days 1, 8, and 15 of about a 4-week cycle.
160. The use of any one of embodiments 146 or 151-154, wherein the antibody-drug conjugate is administered on days 1, 8, and 15 of a 4-week cycle.
161. The use of any one of embodiments 146-160, wherein the subject has been previously treated with one or more therapeutic agents and did not respond to the treatment, wherein the one or more therapeutic agents is not the antibody-drug conjugate.
162. The use of any one of embodiments 146-160, wherein the subject has been previously treated with one or more therapeutic agents and relapsed after the treatment, wherein the one or more therapeutic agents is not the antibody-drug conjugate.
163. The use of any one of embodiments 146-160, wherein the subject has been previously treated with one or more therapeutic agents and has experienced disease progression during treatment, wherein the one or more therapeutic agents is not the antibody-drug conjugate.
164. The use of any one of embodiments 146-163, wherein the subject has been previously treated with a platinum-based therapy.
165. The use of embodiment 164, wherein the cancer is platinum-resistant.
166. The use of embodiment 165, wherein the subject experienced disease progression or relapsed 2 or more months after treatment with the platinum-based therapy.
167. The use of embodiment 165, wherein the subject experienced disease progression or relapsed within 6 months after treatment with the platinum-based therapy.
168. The use of embodiment 165, wherein the subject experienced disease progression or relapsed between 2 months and 6 months after treatment with the platinum-based therapy.
169. The use of any one of embodiments 164-168, wherein cancer is not platinum-refractory.
170. The use of any one of embodiments 164-169, wherein the subject did not experience disease progression or relapse within 2 months after treatment with the platinum-based therapy.
171. The use of any one of embodiments 146-170, wherein the subject has been previously treated with a VEGF antagonist.
172. The use of embodiment 171, wherein the VEGF antagonist is an anti-VEGF antibody.
173. The use of embodiment 172, wherein the anti-VEGF antibody is bevacizumab.
174. The use of any one of embodiments 146-173, wherein the subject received prior systemic therapy and experienced disease progression on or after the systemic therapy.
175. The use of any one of embodiments 146-174, wherein the subject received 1, 2, 3, 4 or 5 rounds of prior systemic therapy.
176. The use of embodiment 175, wherein the prior systemic therapy is a chemotherapy regimen and wherein poly ADP ribose polymerase (PARP) inhibitors are not chemotherapy.
177. The use of any one of embodiments 146-176, wherein the cancer is ovarian cancer.
178. The use of embodiment 177, wherein the ovarian cancer is epithelial ovarian cancer.
179. The use of any one of embodiments 146-176, wherein the cancer is peritoneal cancer.
180. The use of embodiment 179, wherein the peritoneal cancer is primary peritoneal cancer.
181. The use of any one of embodiments 146-176, wherein the cancer is fallopian tube cancer.
182. The use of any one of embodiments 146-181, wherein the cancer is an advanced stage cancer.
183. The use of embodiment 182, wherein the advanced stage cancer is a stage 3 or stage 4 cancer.
184. The use of embodiment 182 or embodiment 183, wherein the advanced stage cancer is metastatic cancer.
185. The use of any one of embodiments 146-184, wherein the cancer is recurrent cancer.
186. The use of any one of embodiments 146-185, wherein the monomethyl auristatin is monomethyl auristatin E (MMAE).
187. The use of any one of embodiments 146-186, wherein the anti-TF antibody or antigen-binding fragment thereof of the antibody-drug conjugate is a monoclonal antibody or a monoclonal antigen-binding fragment thereof.
188. The use of any one of embodiments 146-187, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1.

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6.

189. The use of any one of embodiments 146-188, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
190. The use of any one of embodiments 146-189, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
191. The use of any one of embodiments 146-190, wherein the anti-TF antibody of the antibody-drug conjugate is tisotumab.
192. The use of any one of embodiments 146-191, wherein the antibody-drug conjugate further comprises a linker between the anti-TF antibody or antigen-binding fragment thereof and the monomethyl auristatin.
193. The use of embodiment 192, wherein the linker is a cleavable peptide linker.
194. The use of embodiment 193, wherein the cleavable peptide linker has a formula: -MC-vc-PAB-, wherein:

a) MC is:

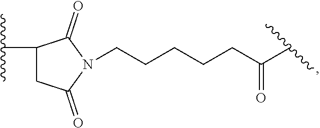 [SMILES: *C(=O)CCCCCN1C(=O)CC(*)C1=O]

b) vc is the dipeptide valine-citrulline, and

c) PAB is:

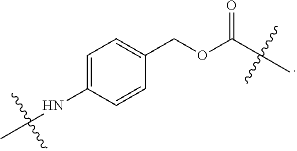 [SMILES: *Nc1ccc(COC(*)=O)cc1]

195. The use of any one of embodiments 192-194, wherein the linker is attached to sulphydryl residues of the anti-TF antibody obtained by partial reduction or full reduction of the anti-TF antibody or antigen-binding fragment thereof.
196. The use of embodiment 195, wherein the linker is attached to monomethyl auristatin E (MMAE), wherein the antibody-drug conjugate has the following structure:

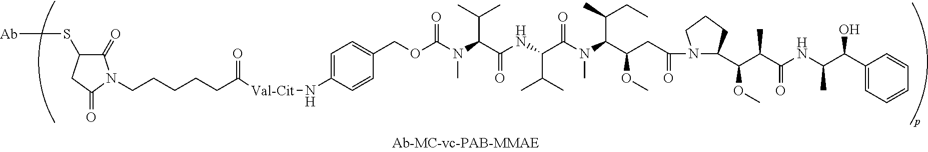

wherein p denotes a number from 1 to 8. S represents a sulphydryl residue of the anti-TF antibody, and Ab designates the anti-TF antibody or antigen-binding fragment thereof.
197. The use of embodiment 196, wherein the average value of p in a population of the antibody-drug conjugates is about 4.
198. The use of any one of embodiments 146-197, wherein the antibody-drug conjugate is tisotumab vedotin.
199. The use of any one of embodiments 146-198, wherein the route of administration for the antibody-drug conjugate is intravenous.
200. The use of any one of embodiments 146-199, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.
201. The use of any one of embodiments 146-200, wherein one or more therapeutic effects in the subject is improved after administration of the antibody-drug conjugate relative to a baseline.
202. The use of embodiment 201, wherein the one or more therapeutic effects is selected from the group consisting of: size of a tumor derived from the cancer, objective response rate, duration of response, time to response, progression free survival, overall survival and CA-125 level.
203. The use of any one of embodiments 146-202, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.
204. The use of any one of embodiments 146-203, wherein the objective response rate is at least about 20%, at least about 25%, at least about 30%, at least about 35%, at least about 40%, at least about 45%, at least about 50%, at least about 60%, at least about 70%, or at least about 80%.
205. The use of any one of embodiments 146-204, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
206. The use of any one of embodiments 146-205, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
207. The use of any one of embodiments 146-206, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
208. The use of any one of embodiments 146-207, wherein the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample obtained from the subject before administration of the antibody-drug conjugate.
209. The use of any one of embodiments 146-208, wherein the subject has one or more adverse events and is further administered an additional therapeutic agent to eliminate or reduce the severity of the one or more adverse events.
210. The use of any one of embodiments 146-209, wherein the subject is at risk of developing one or more adverse events and is further administered an additional therapeutic agent to prevent or reduce the severity of the one or more adverse events.
211. The use of embodiment 209 or embodiment 210, wherein the one or more adverse events is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, or general physical health deterioration.
212. The use of any one of embodiments 209-211, wherein the one or more adverse events is a grade 3 or greater adverse event.
213. The use of nay one of embodiments 209-211, wherein the one or more adverse events is a serious adverse event.
214. The method of any one of embodiments 209-213, wherein the one or more adverse events is conjunctivitis and/or keratitis and the additional agent is a preservative-free lubricating eye drop, an ocular vasoconstrictor and/or a steroid eye drop.
215. The use of any one of embodiments 146-214, wherein the antibody-drug conjugate is administered as a monotherapy.
216. The use of any one of embodiments 146-215, wherein the subject is a human.
217. The use of any one of embodiments 146-216, wherein the antibody-drug conjugate is in a pharmaceutical composition comprising the antibody-drug conjugate and a pharmaceutical acceptable carrier.

### EXAMPLES

### Example 1: Phase I/II Clinical Study of Tisotumab Vedotin Treatment in Subjects with Relapsed, Advanced and/or Metastatic Cancer

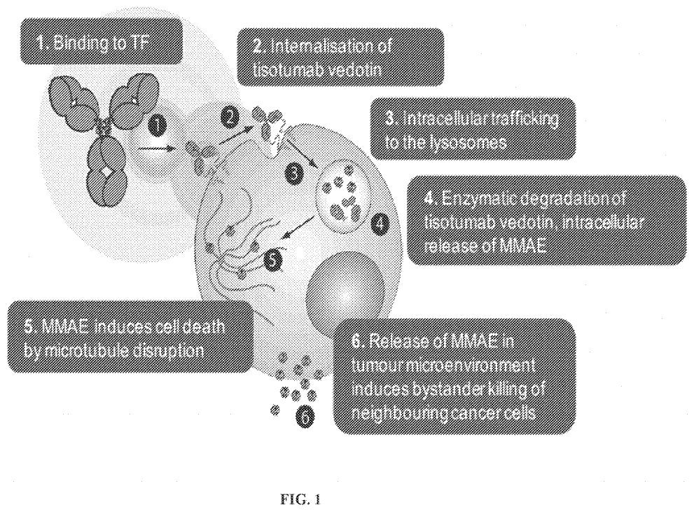

Tisotumab vedotin is an antibody-drug conjugate comprising a TF-targeted human monoclonal immunoglobulin G1 (subtype κ) conjugated via a protease-cleavable valine citrulline linker to the drug monomethyl auristatin E (MMAE), a dolastatin 10 analog. High, differential levels of TF have been observed on the membranes of neoplastic cells as well as on tumor-associated endothelium in multiple cancers. Tisotumab vedotin selectively targets TF to deliver a clinically validated toxic payload to tumor cells (FIG. 1). See Breij E C et al. Cancer Res. 2014; 74(4):1214-1226 and Chu A J. Int J Inflam. 2011, 2011: Article ID 367284; doi: 10.4061/2011/367284. Dolastatins and auristatins belong to a class of chemotherapies that act as microtubule disrupting agents.

### Methods

A phase I/II single arm, multicenter clinical trial investigated the efficacy, safety and tolerability of 2.0 mg/kg Q3W tisotumab vedotin in patients with relapsed, advanced and/or metastatic cancer. A total of 170 patients were enrolled, of which 36 patients (n=36) had ovarian cancer and received at least 1 dose of tisotumab vedotin. Each eligible patient received intravenous (IV) infusion of tisotumab vedotin at a dose of 2.0 mg/kg on day 1 of each 21-day cycle (i.e., each treatment cycle was 3 weeks (Q3W)).

Lyophilized vials containing 40 mg of tisotumab vedotin were stored in a refrigerator at 2° C. to 8° C. Tisotumab vedotin was reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution had a pH of 6.0. The reconstituted tisotumab vedotin was diluted into a 0.9% NaCl 100 mL infusion bag according to the dose calculated for the patient to receive 2.0 mg/kg tisotumab vedotin. Intravenous infusion was completed within 24 hours after the tisotumab vedotin vial had been reconstituted. A 0.2 μm in-line filter was used for the intravenous infusion. The entire 100 mL volume from the prepared infusion bag was administered. No dead volume was provided.

A primary objective of the study was to assess safety and tolerability of tisotumab vedotin. Adverse event (AE) severity was graded according to CTCAE version 4.03. Secondary objectives of the study included: 1) evaluation of the pharmacokinetic profile of tisotumab vedotin after single and multiple infusions (e.g., after the first 3-week treatment cycle and at the end of the trial with an expected average of 6 months); and (2) preliminary assessment of anti-tumor activity of tisotumab vedotin based on the tumor size and or CA-125 level. Tumor evaluations were performed by CT scans.

The following subjects were eligible for the study: (1) subjects with relapsed, advanced and/or metastatic cancer and had failed available standard treatments, or were not candidates for standard therapy; (2) subjects with measurable diseases; (3) subjects who were at least 18 years old; (4) subjects with acceptable renal function, liver function, hematological status (without hematologic support), and acceptable coagulation status; (5) subjects with Eastern Cooperative Oncology Group (ECOG) performance status of 0 or 1; (6) subjects with life expectancy of at least three months; (7) subjects with negative serum pregnancy test results, if the subjects were female and between 18-55 years old; (8) subjects who were not pregnant or breast feeding women; (9) subjects, both females and males, of reproductive potential that agreed to use adequate contraception during and for six months after the last infusion of tisotumab vedotin; and (10) subjects that provided signed informed consent.

The following subjects were excluded from the study: (1) subjects with known past or current coagulation defects; (2) subjects who had ongoing major bleeding; (3) subjects with clinically significant cardiac diseases; (4) subjects who had a baseline QT interval as corrected by Fridericia's formula (QTcF) of more than 450 msec, a complete left bundle branch block (defined as a QRS interval ≥120 msec in left bundle branch block form) or an incomplete left bundle branch block; (5) subjects who had received granulocyte colony stimulating factor (G-CSF) or granulocyte/macrophage colony stimulating factor support within one week or pegylated G-CSF within two weeks before the Screening Visit; (6) subjects who had received a cumulative dose of corticosteroid of at least 100 mg (prednisone or equivalent doses of corticosteroids) within two weeks before the first infusion; (7) subjects who had major surgery within six weeks or open biopsy within 14 days before drug infusion, or had plan for any major surgery during treatment period; (8) subjects with any history of intracerebral arteriovenous malformation, cerebral aneurysm, brain metastases or stroke; (9) subjects who had any anticancer therapy including small molecules, immunotherapy, chemotherapy monoclonal antibodies or any other experimental drug within four weeks or five half-lives, whichever was longest, before first infusion; (10) subjects who had prior treatment with bevacizumab within twelve weeks before the first infusion; (11) subjects who had radiotherapy within 28 days prior to first dose; (12) subjects who had not recovered from symptomatic side effects of radiotherapy at the time of initiation of screening procedure; (13) subjects with known past or current malignancy other than inclusion diagnosis, except for cervical carcinoma of Stage 1B or less, non-invasive basal cell or squamous cell skin carcinoma, non-invasive, superficial bladder cancer, prostate cancer with a current PSA level <0.1 ng/mL, or any curable cancer with a complete response (CR) of more than 5 years duration; (14) subjects with known human immunodeficiency virus seropositivity; (15) subjects with positive serology (unless due to vaccination or passive immunization due to Ig therapy) for hepatitis B; (16) subjects with positive serology for hepatitis C based on test at screening; (17) subjects with inflammatory bowel disease including Crohn's disease and colitis ulcerosa; (18) subjects with inflammatory lung disease including moderate and severe asthma and chronic obstructive pulmonary disease (COPD) requiring chronic medical therapy; or (19) subjects with ongoing acute or chronic inflammatory skin disease.

### Results

Among the 36 ovarian cancer patients treated with tisotumab vedotin, the objective response rate (ORR) was 17% (6 patients) with 3 confirmed responses (8%).

### Example 2: Phase I/II Clinical Study of Dose-Dense Tisotumab Vedotin Treatment in Subjects with Relapsed, Advanced and/or Metastatic Cancer

### Methods

A phase I/II single arm, multicenter clinical trial investigated the efficacy, safety and tolerability of 1.2 mg/kg 3Q4W tisotumab vedotin in patients with recurrent, advanced and/or metastatic cancer. A total of 24 patients were enrolled, of which 12 patients (n=12) had ovarian cancer and received at least 1 dose of tisotumab vedotin. Each eligible patient received intravenous (IV) infusion of tisotumab vedotin at a dose of 1.2 mg/kg on days 1, 8 and 15 of each 28-day cycle (i.e., each treatment cycle was 4 weeks (3Q4W); also referred herein as “dose-dense schedule”). One patient was switched onto 2.0 mg/kg tisotumab vedotin Q3W beginning at Cycle 6, after her response had been recorded.

Lyophilized vials containing 40 mg of tisotumab vedotin were stored in a refrigerator at 2° C. to 8° C. Tisotumab vedotin was reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution had a pH of 6.0. The reconstituted tisotumab vedotin was diluted into a 0.9% NaCl 100 mL infusion bag according to the dose calculated for the patient to receive 1.2 mg/kg tisotumab vedotin. Intravenous infusion was completed within 24 hours after the tisotumab vedotin vial had been reconstituted. A 0.2 μm in-line filter was used for the intravenous infusion. The entire 100 mL volume from the prepared infusion bag was administered. No dead volume was provided.

A primary objective of the study was to assess safety and tolerability of tisotumab vedotin. Adverse events were measured throughout the trial from the first treatment until the end of the trial. Adverse event (AE) severity was graded according to CTCAE version 4.03. Secondary objectives of the study included assessment of the pharmacokinetic profile of tisotumab vedotin and preliminary assessment of the efficacy of the dose-dense regimen of tisotumab vedotin in treating ovarian cancer. Secondary outcome measures included: 1) area under the curve (AUC) of tisotumab vedotin; 2) maximum of plasma concentration of tisotumab vedotin; 3) half-life of tisotumab vedotin; 4) free toxin level (i.e., MMAE); 5) clinical response of the subjects according to RECIST version 1.1 criteria; and 6) response evaluation based on CA125 level.

The following subjects were eligible for the study: (1) subjects with relapsed, advanced and/or metastatic cancer and had failed available standard treatments, or were not candidates for standard therapy; (2) subjects with measurable diseases; (3) subjects who were at least 18 years old; (4) subjects with acceptable renal function, liver function, hematological status (without hematologic support), and acceptable coagulation status; (5) subjects with ECOG performance status of 0 or 1; (6) subjects with life expectancy of at least three months; (7) subjects with negative serum pregnancy test results, if the subjects were female and between 18-55 years old; (8) subjects who were not pregnant or breast feeding women; (9) subjects, both females and males, of reproductive potential that agreed to use adequate contraception during and for six months after the last infusion of tisotumab vedotin; and (10) subjects that provided signed informed consent.

The following subjects were excluded from the study: (1) subjects with known past or current coagulation defects; (2) subjects who had ongoing major bleeding; (3) subjects with clinically significant cardiac diseases; (4) subjects who had a baseline QT interval as corrected by Fridericia's formula (QTcF) of more than 450 msec, a complete left bundle branch block (defined as a QRS interval ≥120 msec in left bundle branch block form) or an incomplete left bundle branch block; (5) subjects who had therapeutic anti-coagulative or long term anti-platelet treatments; (6) subjects who had received granulocyte colony stimulating factor (G-CSF) or granulocyte/macrophage colony stimulating factor support within one week or pegylated G-CSF within two weeks before the Screening Visit; (7) subjects who had received a cumulative dose of corticosteroid of at least 100 mg (prednisone or equivalent doses of corticosteroids) within two weeks before the first infusion; (8) subjects who had dietary supplements during the study period, except multivitamins, vitamin D and calcium; (9) subjects who had major surgery within six weeks or open biopsy within 14 days before drug infusion, or had plan for any major surgery during treatment period; (10) subjects with any history of intracerebral arteriovenous malformation, cerebral aneurysm, brain metastases or stroke; (11) subjects who had any anticancer therapy including small molecules, immunotherapy, chemotherapy monoclonal antibodies or any other experimental drug within four weeks or five half-lives, whichever was longest, before first infusion; (12) subjects who had prior treatment with bevacizumab within twelve weeks before the first infusion; (13) subjects who had prior therapy with a conjugated or unconjugated auristatin derivative; (14) subjects who had radiotherapy within 28 days prior to first dose; (15) subjects who had not recovered from symptomatic side effects of radiotherapy at the time of initiation of screening procedure; (16) subjects with known past or current malignancy other than inclusion diagnosis, except for cervical carcinoma of Stage 1B or less, non-invasive basal cell or squamous cell skin carcinoma, non-invasive, superficial bladder cancer, prostate cancer with a current PSA level <0.1 ng/mL, breast cancer in BRCA1 or BRACA2 positive ovarian cancer, or any curable cancer with a complete response (CR) of more than 5 years duration; (17) subjects with radiographic evidence of cavitating pulmonary lesions and tumor adjacent to or invading any large blood vessel unless approved by sponsor; (18) subjects with ongoing, significant, uncontrolled medical conditions; (19) subjects with peripheral neuropathy; (20) subjects with active viral, bacterial or fungal infections requiring intravenous treatment with antimicrobial therapy starting less than four weeks prior to first dose; (21) subjects who received oral treatment with antimicrobial therapy starting less than two weeks prior to first dose; (22) subjects with known human immunodeficiency virus seropositivity; (22) subjects with positive serology (unless due to vaccination or passive immunization due to Ig therapy) for hepatitis B; (23) subjects with positive serology for hepatitis C based on test at screening; (24) subjects with inflammatory bowel disease including Crohn's disease and colitis ulcerosa; (25) subjects with inflammatory lung disease including moderate and severe asthma and chronic obstructive pulmonary disease (COPD) requiring chronic medical therapy; or (26) subjects with ongoing acute or chronic inflammatory skin disease.

### Results

Among the 12 ovarian cancer patients who received tisotumab vedotin with the dose-dense schedule, the objective response rate (ORR) was 33% (4 patients). Two of these 4 responses were confirmed. Compared to the results of the clinical trial described in Example 1, in which patients received 2.0 mg/kg Q3W tisotumab vedotin treatment, the limited data in this clinical trial suggests that efficacy of tisotumab vedotin may be improved with a dose-dense schedule as compared to the Q3W schedule in patients with ovarian cancer.

### Example 3: A Phase II Study of Tisotumab Vedotin in Subjects with Platinum-Resistant Ovarian Cancer

This study evaluates the efficacy, safety and tolerability of 2.0 mg/kg Q3W and 0.9 mg/kg 3Q4W (“dose-dense regimen”) tisotumab vedotin in patients with epithelial ovarian cancer, primary peritoneal cancer, or fallopian tube cancer that has relapsed within 6 months of the completion of platinum-based treatment.

Despite initial therapy, the vast majority of women with ovarian cancer will relapse and require subsequent therapy. Platinum-free interval is a strong predictor of treatment success in recurrent ovarian cancer (Pujade-Lauraine E. and Alexandre J., Ann. Oncol. 22 Suppl. 8: viii 61-4 (2011)). Patients whose disease relapses within 6 months after platinum-containing therapy are categorized as having platinum-resistant disease. At first relapse, approximately 25% of the patients have platinum-resistant ovarian cancer (PROC), and the vast majority of patients with recurrent disease will eventually develop PROC (Slaughter K. et al., Gynecol. Oncol. 142(2): 225-30 (2016)). For most PROC patients, single agent chemotherapy rather than combination therapy is favored in the first line. Single agents approved for PROC have overall RECIST response rates around 12% and progression-free survival (PFS) around 3.4 months (Pujade-Lauraine E. et al., J. Clin. Oncol. 32(13): 1302-8 (2014)). For patients who relapse after first-line therapy for PROC and are fit enough to receive subsequent treatment there is no standard of care. Clinical benefit, as measured by PFS and overall survival (OS), diminishes significantly below even the poor prognosis of first line treatment as the line of therapy increases (Hanker L C et al., Ann. Oncol. 23(10): 2605-12 (2012)).

### Methods

This randomized, open label, multicenter trial is designed to evaluate the safety, antitumor activity, and pharmacokinetics of tisotumab vedotin (TV) administered every 3 weeks (Q3W) or on days 1, 8, and 15 of every 4-week cycle (3Q4W [dose-dense regimen]) for the treatment of various types of platinum-resistant ovarian cancer. The study has an initial safety run-in period followed by a phase 2 period.

Eligible patients are at least 18 years of age, must have PROC, be eligible for single agent chemotherapy, and must have previously received a bevacizumab-containing treatment regimen for ovarian cancer, if eligible. Safety run-in patients may have received up to 5 prior systemic treatment regimens for ovarian cancer. Phase 2 patients must have received at most 1 prior cytotoxic chemotherapy regimen in the PROC setting. Approximately 142 patients may be enrolled in the study. This includes 6-12 patients in the safety run-in phase as well as approximately 30 patients in each of two phase 2 cohorts with a possible expansion of up to approximately 70 additional patients in one of the two phase 2 cohorts.

Inclusion criteria and exclusion criteria for patients enrolled in trial are shown in Table 1.

TABLE 1
List of inclusion and exclusion criteria

| Inclusion | Histologic documentation of epithelial ovarian cancer, primary peritoneal cancer, | |
|---|---|---|
| Criteria | or fallopian tube cancer (excluding carcinosarcoma, mucinous, and low grade | |
| | serous histologies), hereafter referred to as “ovarian cancer”. | |
| | If eligible, patients must have received previous treatment with a bevacizumab- | |
| | containing regimen for ovarian cancer. Prior bevacizumab may have been | |
| | given at any line of treatment. | |
| | Safety run-in patients only: Platinum-resistant ovarian cancer (PROC), which is | |
| | defined as having progressed or relapsed within 6 months after previous | |
| | platinum-containing chemotherapy and for which single agent chemotherapy | |
| | is appropriate. Progression or relapse must be documented radiographically | |
| | using RECIST v1.1 criteria. The patient may have received up to 5 prior | |
| | systemic treatment regimens for ovarian cancer. | |
| | Phase 2 patients only: PROC. The patient must have received at most 1 prior | |
| | cytotoxic chemotherapy regimen in the PROC setting. Patients eligible to | |
| | receive a PARP inhibitor may have received such therapy; PARP inhibitors | |
| | are not considered cytotoxic chemotherapy regimens for the purposes of this | |
| | study. | |
| | Measurable disease according to RECIST v1.1 as assessed by the investigator. | |
| | a. | A minimum of one non-nodal lesion ≥10 mm in the longest diameter |
| | | from a non-irradiated area. If target lesion(s) are located within |
| | | previously irradiated area only, the patient can be enrolled only if there |
| | | has been demonstrated progression in the “in field” lesion and upon |
| | | approval of the sponsor's medical monitor. |
| | b. | Lymph node lesion ≥15 mm in the shortest diameter from a non- |
| | | irradiated area. |

| | |
|---|---|
| | Age 18 years or older. |
| | An Eastern Cooperative Oncology Group (ECOG) Performance Status score of 0 |
| | or 1. |
| | The following baseline laboratory data: |
| | Absolute neutrophil count (ANC) ≥1500/μL assessed at least 2 weeks after |
| | growth factor support, if applicable. |
| | Platelet count ≥100 × 10⁹/L assessed at least 2 weeks after transfusion with |
| | blood products. |
| | Hemoglobin ≥5.6 mmol/L (9.0 g/dL) assessed at least 2 weeks after |
| | transfusion with blood products. |
| | Serum bilirubin ≤1.5 × upper limit of normal (ULN) or direct bilirubin ≤2 × |
| | ULN in patients diagnosed with Gilbert's syndrome. |
| | Estimated glomerular filtration rate (eGFR) ≥60 mL/min/1.73m² using the |
| | Modification of Diet in Renal Disease (MDRD) study equation as applicable |
| | Alanine aminotransferase (ALT) and aspartate aminotransferase (AST) ≤2.5 × |
| | ULN. (If liver tumor/metastases are present, then <5 × ULN is allowed). |
| | Acceptable coagulation status: |
| | INR ≤1.2 without anticoagulation therapy. |
| | aPTT ≤1.25 ULN. |
| | Life expectancy of at least 3 months. |
| | Patients of childbearing potential, under the following conditions: |
| | Must have a negative serum or urine pregnancy test (minimum sensitivity |
| | 25 mIU/mL or equivalent units of beta human chorionic gonadotropin [β- |
| | hCG]) result within 7 days prior to the first dose of tisotumab vedotin. |
| | Patients with false positive results and documented verification that the |
| | patient is not pregnant are eligible for participation. |
| | Must agree not to try to become pregnant during the study and for at least |
| | 6 months after the final dose of study drug administration. |
| | Must agree not to breastfeed or donate ova, starting at time of informed |
| | consent and continuing through 6 months after the final dose of study drug |
| | administration. |
| | If sexually active in a way that could lead to pregnancy, must consistently use |
| | 2 highly effective methods of birth control starting at time of informed |
| | consent and continuing throughout the study and for at least 6 months after |
| | the final dose of study drug administration. |
| | Able to provide fresh tissue for biomarker analysis from a newly obtained core or |
| | excisional biopsy of a tumor lesion. If available, archived tumor tissue is also |
| | requested for additional biomarker analysis. |
| | Note: Patients for whom fresh samples cannot be obtained (e.g., inaccessible |
| | tumor or patient safety concerns) may submit an archived specimen in place |
| | of the fresh tissue at baseline upon agreement from the sponsor's medical |
| | monitor. |
| | The patient or the patient's legally authorized representative must provide written |
| | informed consent. |
| Exclusion | 1. Primary platinum-refractory disease, defined as disease progression within 2 |
| Criteria | months of completion of first line platinum-based therapy. |
| | Patients with clinical symptoms or signs of gastrointestinal obstruction within the |
| | past 6 months or who currently require parenteral nutrition. |
| | Hematological: Known past or current coagulation defects leading to an |
| | increased risk of bleeding; diffuse alveolar hemorrhage from vasculitis; |
| | known bleeding diathesis; ongoing major bleeding; trauma with increased |
| | risk of life-threatening bleeding or history of severe head trauma or |
| | intracranial surgery within 8 weeks of trial entry. |
| | Cardiovascular: Clinically significant cardiac disease including uncontrolled |
| | hypertension (systolic BP >150 mmHg or diastolic BP >90 minHg), |
| | unstable angina, acute myocardial infarction within 6 months prior to |
| | screening, serious cardiac arrhythmia requiring medication (not including |
| | asymptomatic atrial fibrillation with controlled ventricular rate); any medical |
| | history of congestive heart failure (Class II or higher as classified by the New |
| | York Heart AssociationError! Reference source not found.), or any |
| | medical history of decreased cardiac ejection fraction of <45%. |
| | Ophthalmological: Active ocular surface disease at baseline. An ocular |
| | evaluation is to be confirmed by an ophthalmologist at screening. Patients |
| | with any prior episode of cicatricial conjunctivitis or Steven Johnson |
| | syndrome (as evaluated by the investigator) are ineligible. |
| | History of another malignancy within 3 years before the first dose of study drug, |
| | or any evidence of residual disease from a previously diagnosed malignancy. |
| | Exceptions are malignancies with a negligible risk of metastasis or death |
| | (e.g., 5-year overall survival ≥90%), such as adequately treated carcinoma in |
| | situ of the cervix, non-melanoma skin carcinoma, ductal carcinoma in situ, or |
| | stage I uterine cancer. |
| | Inflammatory bowel disease including Crohn's disease and ulcerative colitis. |
| | Ongoing, acute, or chronic inflammatory skin disease. |
| | Uncontrolled tumor-related pain. |
| | Inflammatory lung disease, including moderate and severe asthma and chronic |
| | obstructive pulmonary disease, requiring chronic medical therapy. |
| | Grade 3 or higher pulmonary disease unrelated to underlying malignancy. |
| | Patients with significant peripheral vascular disease. |
| | Uncontrolled pleural or pericardial effusions. |
| | Medications or treatment regimens: |
| | Therapeutic anticoagulation therapy is not permitted. Upon review of |
| | safety data by the SMC and the sponsor's Drug Safety department after |
| | the first 16 patients have received 2 cycles of tisotumab vedotin |
| | treatment, anticoagulation therapy may be permitted under certain |
| | conditions. |
| | Chronic use of anti-platelet therapy (ASA [aspirin], clopidogrel, and |
| | similar medications), as required for vascular diseases such as coronary |
| | artery disease, cerebrovascular accident, and similar conditions, is not |
| | permitted on this study. |
| | Cumulative dose of corticosteroid ≥150 mg (prednisone or equivalent |
| | doses of corticosteroids) within 2 weeks of the first tisotumab vedotin |
| | administration is prohibited. |
| | Surgery/procedures: Major surgical procedure (defined as a surgery requiring |
| | inpatient hospitalization of at least 48 hours) within 4 weeks or excisional |
| | biopsy within 7 days prior to the first study drug administration. Patients who |
| | have planned major surgery during the treatment period must be excluded |
| | from the trial. |
| | Received a live vaccine within 30 days prior to the first dose of trial treatment. |
| | Examples of live vaccines include, but are not limited to, the following: |
| | measles, mumps, rubella, varicella/zoster (chicken pox), yellow fever, rabies, |
| | Bacillus Calmette-Guérin, and typhoid vaccine. Seasonal influenza vaccines |
| | for injection are generally killed virus vaccines and are allowed; however, |
| | intranasal influenza vaccines (e.g., FLUMIST ®) are live attenuated vaccines |
| | and are not allowed. |
| | Grade >1 peripheral neuropathy from any cause. |
| | Prior therapy: |
| | Any prior treatment with MMAE-derived drugs. |
| | Radiotherapy within 21 days prior to the first administration of study |
| | drug. Patients must have recovered from all radiation-related toxicities. |
| | At least 42 days must have elapsed from the last administration of chemo- |
| | radiotherapy. |
| | Small molecules, chemotherapy, immunotherapy, biologics, experimental |
| | agents, or any other antitumor therapy within 21 days prior to the first |
| | administration of study drug. If underlying disease is progressing on |
| | treatment, patients may enroll within 21 days upon approval of the |
| | sponsor's medical monitor. These patients must have recovered from all |
| | related toxicities. |
| | Any uncontrolled Grade 3 or higher per the NCI CTCAE v5.0) viral, bacterial, |
| | or fungal infection within 2 weeks prior to the first dose of tisotumab vedotin. |
| | Routine antimicrobial prophylaxis is permitted. |
| | Known seropositivity of human immunodeficiency virus; known medical history |
| | of Hepatitis B or C infection. |
| | Note: No testing for human immunodeficiency virus, Hepatitis B, or |
| | Hepatitis C is required unless mandated by local health authorities. |
| | Known history of untreated brain metastasis or active brain metastasis. Patients |
| | with symptoms of brain metastasis should be screened for this condition prior |
| | to enrollment. |
| | Patients who are breastfeeding, pregnant, or planning to become pregnant from |
| | time of informed consent until 6 months after final dose of study drug |
| | administration. |
| | Known hypersensitivity to any excipient contained in the drug formulation of |
| | tisotumab vedotin. |
| | Other serious underlying medical condition that, in the opinion of the |
| | investigator, would impair the patient's ability to receive or tolerate the |
| | planned treatment and follow-up. |

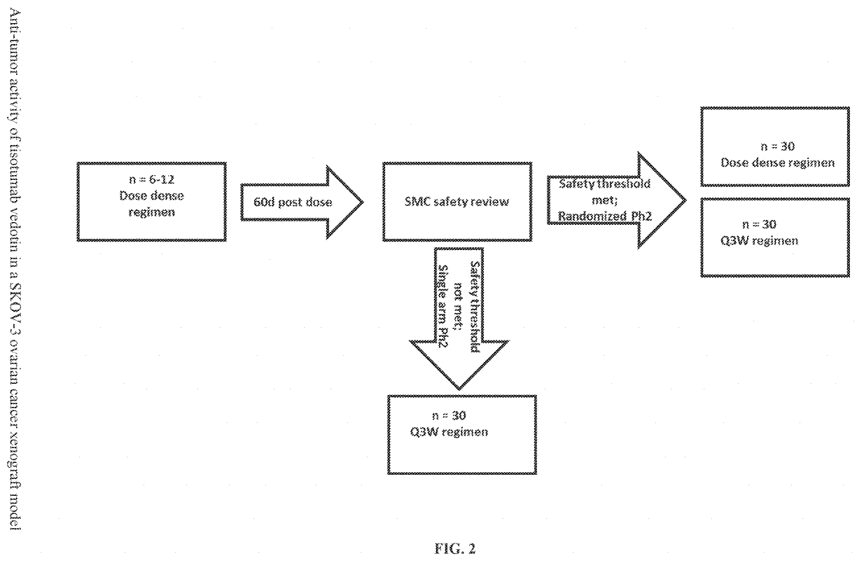

FIG. 2 shows a schematic of the study design. The safety run-in period evaluates the safety of the dose-dense regimen in at least 6 patients. If no more than 1 patient experiences a DLT among all safety run-in patients given 0.9 mg/kg on a dose-dense schedule, patients will be randomized in the phase 11 portion to receive tisotumab vedotin 2.0 mg/kg intravenously (IV) every 3 weeks (Q3W regimen) or 0.9 mg/kg on Days 1, 8, and 15 of every 4-week cycle (dose-dense regimen). If dose-limiting DLTs are experienced by 2 or more patients at 0.9 mg/kg during the safety run-in portion, the dose of tisotumab vedotin will be de-escalated to 0.65 mg/kg and 6 more patients will be enrolled at this dose on the dose-dense schedule. If an additional two more patients experience DLTs with the dose-dense regimen at 0.65 mg/kg tisotumab vedotin, the phase 2 portion of the study will be a single arm study of the Q3W regimen.

Based on safety data collected up to 60 days from start of treatment, the study will proceed to randomization in the phase II portion of the study unless DLTs occur as described above or other unacceptable toxicities are observed. All Grade 3 or higher non-ophthalmologic Adverse Events (AEs) are reviewed with the Safety Monitoring Committee (SMC) to assess tolerability and safety of tisotumab vedotin. If tolerability of the dose-dense schedule is confirmed, patients will be randomized in a 1:1 ratio to receive 2.0 mg/kg TV every 3 weeks (Q3W) or the dose-dense regimen determined during the safety run-in (0.9 mg/kg or 0.65 mg/kg on Days 1, 8, and 15 of every 4-week cycle). Randomization will be stratified by first line vs. second line PROC and histology (serous vs. non-serous). Tisotumab vedotin dosing is capped at the 100 kg patient weight equivalent in all cohorts. If tolerability of the dose-dense schedule is not confirmed after the safety run-in portion, the phase 2 portion will consist of a single arm study of the Q3W dosing regimen.

Lyophilized vials containing 40 mg of tisotumab vedotin are stored in a refrigerator at 2° C. to 8° C. Tisotumab vedotin is reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution has a pH of 6.0. The reconstituted tisotumab vedotin is diluted into a 0.9% NaCl 100 mL infusion bag according to the dose calculated for the patient. Intravenous infusion is completed within 24 hours after the tisotumab vedotin vial had been reconstituted. A 0.2 μm in-line filter is used for the intravenous infusion. The entire 100 mL volume from the prepared infusion bag is administered. No dead volume is provided. The infusion is given over approximately 30 minutes in the absence of infusion-related reactions (IRRs).

For patients who do not tolerate the protocol-specified dosing schedule, dose reductions are permitted in order to allow the patient to continue tisotumab vedotin treatment according to the dose modification scheme in Table 2.

TABLE 2
Dose Modification Scheme

| Dose-dense regimen | | Q3W regimen | |
|---|---|---|---|
| Previous dose | Reduced dose | Previous dose | Reduced dose |
| 0.9 mg/ka (90 | 0.65 mg/kg (65 | 2.0 mg/kg (200 | 1.3 mg/kg (130 |
| mg maximum | mg maximum | mg maximum | mg maximum |
| total dose) on | total dose) on | total dose | total dose) |
| D1, D8, and D15 | D1, D8, and D15 | | |
| 0.65 mg/kg (65 | 0.65^{#} mg/kg (65 | 1.3 mg/kg (130 | 0.9* mg/kg (90 |
| mg maximum | mg maximum | mg maximum | mg maximum |
| total dose) on | total dose) on | total dose) | total dose) |
| D1, D8, and D15 | D1 and D8 | | |

^{#}If the patient is already being treated with tisotumab vedotin 0.65 mg/kg on D1 and D8, the dose must not be reduced further.
*If the patient is already being treated with tisotumab vedotin 0.9 mg/kg, the dose of tisotumab vedotin is not reduced further.

Objectives and endpoints are described in Table 3. The confirmed objective response rate (ORR) is defined as the proportion of patients who achieve a confirmed CR or PR according to RECIST v1.1 as assessed by the investigator. Patients who do not have at least 2 post-baseline response assessments (initial response and confirmation scan) are counted as non-responders.

Confirmed and unconfirmed ORR is defined as the proportion of patients who achieve a CR or PR according to RECIST v1.1 as assessed by the investigator. These include patients with confirmed responses as well as those whose responses were not confirmed or had not yet been assessed for confirmation. DOR is defined as the time from the first documentation of objective response (CR or PR that is subsequently confirmed) to the first documentation of PD or death due to any cause, whichever comes first. Patients who do not have at least 1 post-baseline response assessment are counted as non-responders.

CA-125 response rate is defined as the proportion of patients who have at least a 50% reduction in CA-125 value from baseline. The response must be confirmed in a subsequent sample collected ≥28 days after the prior sample. The absolute value of the confirmatory sample must be ≤10% of the prior sample. Only patients who have an elevated baseline CA-125 value of ≥2×ULN and within 2 weeks prior to the first dose of study drug are included in the analysis. The combined RECIST/CA-125 overall response is defined as the proportion of patients whose best response is a CR or PR according to the Gynecological Cancer Intergroup (GCIG) combined RECIST and CA-125 criteria (Rustin G J. et al., Int. J. Gynecol. Cancer 21(2): 419-23 (2011)).

DCR is defined as the proportion of patients who achieve a CR or PR according to RECIST v1.1 as assessed by the investigator, or meet the SD criteria at least once after start of study treatment at a minimum interval of 12 weeks (−1 week window). Patients who do not have at least 1 post-baseline response assessment are counted as non-responders.

DOR is defined as the time from the first documentation of objective response (CR or PR that is subsequently confirmed) to the first documentation of PD or death due to any cause, whichever comes first. DOR is only calculated for patients who achieve a confirmed CR or PR according to RECIST v1.1 as assessed by the investigator.

TTR is defined as the time from the start of study treatment to the first documentation of objective response (CR or PR that is subsequently confirmed). TTR is only calculated for patients who achieve a confirmed CR or PR, and is summarized with descriptive statistics.

PFS is defined as the time from the start of study treatment to the first documentation of PD or death due to any cause, whichever comes first. Patients who are known to have died and who do not have an evaluation of tumor response after their first dose of study drug are censored at Day 1.

OS is defined as the time from the start of study treatment to date of death due to any cause. In the absence of death, survival time will be censored at the last date the patient is known to be alive (i.e., date of last contact).

The 2-sided 95% exact confidence interval (CI) using the Clopper-Pearson method are calculated for the response rates where applicable (e.g., ORR). For time-to-event endpoints, the median survival time is estimated using the Kaplan-Meier method; the associated 95% CI is calculated based on the complementary log-log transformation.

TABLE 3
Objectives and endpoints

| Primary Objective | Corresponding Primary Endpoint |
|---|---|
| (Safety run-in) Evaluate safety and | Incidence of DLTs or other |
| tolerability of a dose-dense regimen | unacceptable toxicities |
| of tisotumab vedotin | |
| (Phase 2) Evaluate antitumor | Investigator-determined confirmed |
| activity of tisotumab vedotin | ORR as measured by RECIST v1.1 |
| Secondary Objectives (Phase 2) | Corresponding Secondary Endpoints |
| Evaluate the safety and tolerability | Type, incidence, severity, seriousness, |
| of tisotumab vedotin | and relatedness of AEs |
| Evaluate preliminary antitumor | Investigator-determined confirmed and |
| activity of tisotumab vedotin | unconfirmed ORR as measured by |
| | RECIST v 1.1 (all responses) |
| Evaluate antitumor activity of | CA-125 response rate |
| tisotumab vedotin | Combined RECIST/CA-125 overall |
| | response |
| Evaluate durability of response in | Investigator- determined duration of |
| patients who respond to tisotumab | response (DOR) as measured by |
| vedotin | RECIST v1.1 |
| Evaluate stability and control of | Investigator-determined disease |
| disease | control rate (DCR) as measured by |
| | RECIST v1.1 |
| Evaluate the timing of responses | Investigator- determined time to |
| | response (TTR) as measured by |
| | RECIST v1.1 |
| Evaluate progression-free survival | Investigator- determined PFS as |
| (PFS) of patients treated with | measured by RECIST v1.1 |
| tisotumab vedotin | |
| Evaluate survival of patients | Overall survival (OS) |
| treated with tisotumab vedotin | |
| Assess pharmacokinetics of | Selected PK parameters for tisotumab |
| tisotumab vedotin | vedotin and MMAE |
| Assess immunogenicity of | Incidence of antitherapeutic antibodies |
| tisotumab vedotin | (ATAs) to tisotumab vedotin |
| Additional Objectives (Phase 2) | Corresponding Additional Endpoints |
| Evaluate Tissue Factor expression- | TF expression-response relationship |
| response relationship | following treatment with tisotumab |
| | vedotin |
| Assess biomarkers of biological | Relationship between biomarkers in |
| activity and resistance and | blood and tumor tissue to efficacy, |
| predictive biomarkers of | safety, or other biomarker endpoints |
| response | following treatment with tisotumab |
| | vedotin |
| Patient-reported outcomes | PROMIS and an NCI PRO-CTCAE |
| | questionnaire customized to focus on |
| | ocular symptoms, bleeding, and |
| | gastrointestinal symptoms, as well as |
| | other questions added by the sponsor |

Patients continue to receive tisotumab vedotin treatment until disease progression unacceptable toxicity, investigator decision, consent withdrawal, start of a subsequent anticancer therapy, study termination by the sponsor, pregnancy, or death, whichever comes first. Response is assessed every 6 weeks for the first 6 months, every 12 weeks for the next 6 months, and then every 6 months after that. RECIST v1.1 is used by the investigator to score responses for primary and secondary endpoints as well as progression. Objective responses are confirmed with repeat scans 4-6 weeks after the first documentation of response. The study is closed 3 years after the last patient enrolled or when no patients remain in long-term follow-up, whichever comes first. Additionally, the sponsor may terminate the study at any time.

Biomarker analysis is not to be utilized for patient selection. Biomarker assessments in tumor tissue may include, but are not limited to: measurement of TF protein, mRNA expression, disease subtype, tumor immune microenvironment, and tumor mutational load. Assessments in blood may include, but are not limited to: cancer markers such as CA-125, cytokine measurements, abundance and phenotypes of immune cell subsets, and circulating nucleic acids. Methods of analysis may include immunohistochemistry (IHC), PCR and T-cell receptor beta chain sequencing, multiplex immune histofluorescence, mutation and gene expression profiling. Next Generation Sequencing, flow cytometry, and proteomic methodologies such as enzyme-linked immunosorbent assay (ELISA) and microvesicle assessment.

Safety assessments include the surveillance and recording of AEs, physical examination findings, eye examinations, vital signs, electrocardiograms (ECGs), concomitant medications, pregnancy testing, and laboratory tests. Safety assessments are performed while the patient continues to receive treatment. After discontinuation of study treatment, patients are followed every 12 weeks for subsequent cancer therapies and survival.

The primary analysis of the study is performed when all treated patients have been followed for at least 6 months or come off study, whichever comes first. Patients enrolled in the safety run-in and phase 2 portions of the study are summarized separately. Safety measurements is summarized by descriptive statistics based on the safety analysis set. The safety analysis set includes all patients who received any amount of study treatment.

As exploratory analyses, subgroup analyses may be conducted for selected endpoints. Subgroups may include but are not limited to the following: platinum-free interval, histology, first line vs. second line PROC, TF expression, prior treatment with checkpoint inhibitors (CPIs) and prior treatment with PARP inhibitors.

Adverse events of special interest include ocular adverse events, infusion-related reactions, increased bleeding, hemorrhage, elevated liver enzymes, mucositis, neutropenia, and peripheral neuropathy. In order to prevent ocular AEs, the following ocular pre-medication guidelines are followed: (1) Administration of local ocular vasoconstrictor before infusion (brimonidine tartrate or similar) for a total of 72 hours (3 days) after each infusion. Immediately prior to the start of each infusion, 3 drops must be given in each eye. For the next 2 days 1 drop must be administered in each eye, 3 times daily or they may otherwise be used in accordance with the product prescribing information. If the patient does not tolerate ocular vasoconstrictors due to adverse reactions, continued treatment with these drops may be stopped at the discretion of the investigator and following discussion with the sponsor's medical monitor. (2) Application of steroid eye drops (dexamethasone 0.1% eye drops or equivalent) before and after each infusion for a total of 3 days. The first drops are to be given 24 hours prior to start of infusion. Continue treatment for 48 hours thereafter. Steroid eye drops should be administered as 1 drop in each eye, 3 times daily, or used in accordance with the product prescribing information. (3) Use of eye cooling pads during infusion, e.g., Cardinal Health cold packs, refrigerator-based THERA PEARL Eye Mask, or similar. To be applied 5 minutes prior to start of infusion in accordance with the instructions provided with the eye cooling pads. The cooling pads must remain on the patient's eyes during the entire 30-minute infusion and for as long as 30 minutes afterwards. (4) Use of lubricating eye drops during the whole treatment phase of the trial (i.e., from first dose of study drug until 30 days after the last dose of study drug). Frequent use of lubricating eye drops as per standard of care for patients receiving chemotherapy is recommended. Lubricating eye drops should be administered according to the product prescribing information. (5) It is recommended that patients not wear contact lenses while being treated with tisotumab vedotin from the first dose until 30 days after the last dose of study drug.

Tisotumab vedotin may cause Infusion-Related Reactions including severe hypersensitivity or anaphylaxis. Signs and symptoms usually develop during or shortly after drug infusion. In case any clinical significant IRR is observed during or after the first infusion of tisotumab vedotin or at subsequent treatment cycles, the patient should be observed for 2 hours after the end of tisotumab vedotin administration for all subsequent infusions. At all times during infusion, immediate emergency treatment of an anaphylactic reaction according to institutional standards must be assured. In order to treat possible anaphylactic reactions, for instance, dexamethasone 10 mg and epinephrine in a 1:1000 dilution or equivalents must always be available along with equipment for assisted ventilation.

### Example 4: Anti-Tumor Activity of Tisotumab Vedotin in Xenograft Models of Ovarian Cancer

The in vivo anti-tumor efficacy of tisotumab vedotin was tested in xenograft mouse models for ovarian cancer, either derived from a human ovarian cancer cell line (SKOV-3 model) or from a human ovarian cancer tumor specimen (patient-derived xenograft model OVFX 1993).

For the SKOV-3 xenograft model, female immune deficient SCID mice were injected subcutaneously with 5×10⁶ SKOV-3 tumor cells (human ovarian carcinoma cell line, ATCC cat. no. HTB-77), suspended in 200 μl phosphate buffered saline (PBS). The day of tumor inoculation was designated day 0. Tumor volumes were measured at least twice per week using a digital caliper (PLEXX). Tumor volumes (mm³) were calculated as follows: tumor volume=0.52×(length)×(width)². On day 36, when tumors had reached a size of 200-400 mm³, mice were randomized into groups of 7 mice with equal tumor size distribution and treated with by intraperitoneal injection of tisotumab vedotin (1 or 4 mg/kg), an isotype control antibody (the HIV gp120-specific human IgG1 antibody IgG1-b12) conjugated with MMAE (IgG1-b12-MMAE, 4 mg/kg) or an unconjugated isotype control IgG1 (IgG1-b12, 4 mg/kg) diluted in PBS (final volume 100 μl). Treatment was repeated on day 44.

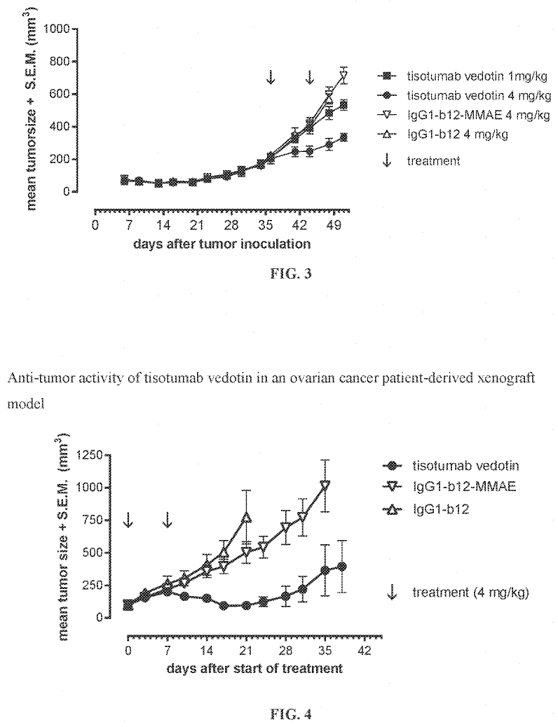

At a dose level of 4 mg/kg, tisotumab vedotin significantly inhibited tumor growth, as demonstrated by reduced tumor size in animals treated with tisotumab vedotin compared to the IgG1-b12 control (p<0.01, Mann-Whitney) but not the isotype control ADC (IgG1-b12-MMAE) (FIG. 3).

The patient-derived xenograft (PDX) ovarian cancer model OVFX 1993 was performed at Oncotest GmbH (Germany). Tumor fragments were removed from donor mice, cut into 4-5 mm fragments and implanted subcutaneously in the flank of athymic nude (NMRI nu/nu) mice, under isofluorane anesthesia. At a tumor volume of 50-250 mm³, mice were randomized and treated intravenously with 4 mg/kg tisotumab vedotin, the isotype control ADC IgG1-b12-MMAE or the unconjugated isotype control antibody IgG -b12 diluted in PBS. The day of randomization and first treatment was designated day 0. A second treatment was administered at day 7. Tumor growth was assessed every 3-4 days by two-dimensional measurement with a caliper. Tumor volumes were calculated according to the following formula: [tumor volume (mm³)=0.5*(a*b²)], in which “a” represents the largest tumor diameter and “b” the perpendicular tumor diameter.

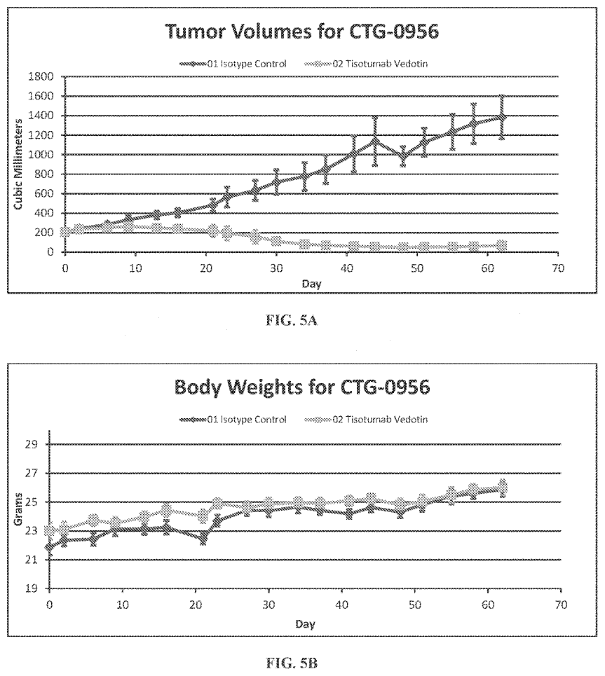

Tisotumab vedotin induced significant anti-tumor activity in the OVFX 1993 ovarian cancer xenograft model compared to the IgG1-b12 control (p<0.01, Dunn's multiple comparison) whereas the isotype control ADC (IgG1-b12-MMAE) did not inhibit tumor growth (FIG. 4).

### Example 5: Anti-Tumor Activity of Tisotumab Vedotin in Xenografts Models of Ovarian Cancer

The in vivo anti-tumor efficacy of tisotumab vedotin was tested in two xenograft mouse models for ovarian cancer from human ovarian cancer tumor specimens (patient-derived xenograft models CTG-0956 and CTG-1086).

Tumors were grown in stock animals until they reached a size of 1.0-1.5 cm³, at which point they were harvested and re-implanted into pre-study animals. Pre-study animals were implanted unilaterally on the left flank with tumor fragments harvested from stock animals. Pre-study tumor volumes were recorded for each experiment beginning seven to ten days after implantation. When tumors reached an average tumor volume of 150-300 mm³ animals were matched by tumor volume into treatment or control groups. Dosing was initiated on Day 0. For each animal model, 8 mice were treated with 2 mg/kg tisotumab vedotin or 2 mg/kg of an isotype control antibody diluted in PBS every 7 days for four doses. Mice were weighed twice weekly and mice exhibiting >20% net weight loss for a period of 7 days or exhibiting >30% net weight loss when compared to Day 0 were euthanized. The study endpoint was when the mean tumor volume of the control group reached 1500 mm³. Beginning on Day 0, animals were observed daily and tumor dimensions were measured twice weekly by digital caliper and data including individual and mean estimated tumor volumes (Mean tumor volume+/−standard error of the mean (SEM) was recorded for each group. Tumor volume was calculated using the formula TV=width²×length×0.52.

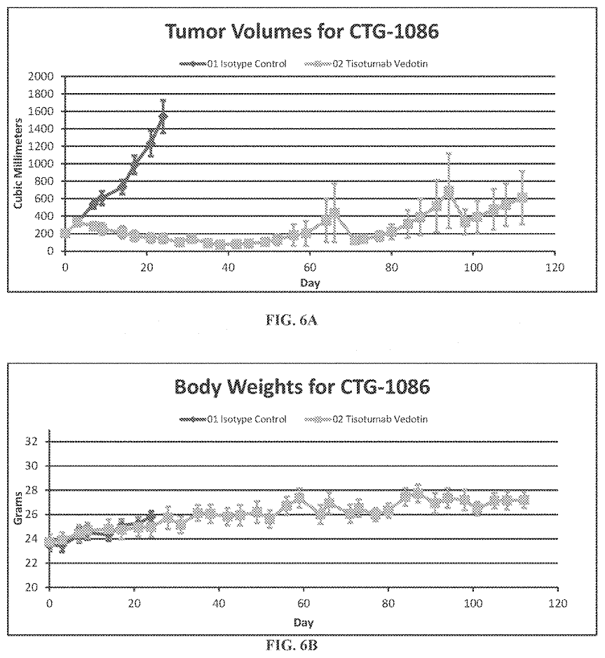

At a dose level of 2 mg/kg, tisotumab vedotin significantly inhibited tumor growth in both the CTG-0956 and CTG-1086 patient-derived xenograft models, as demonstrated by reduced tumor size in animals treated with tisotumab vedotin compared to the isotype control antibody (FIG. 5A and FIG. 6A). Treatment with 2 mg/kg, tisotumab vedotin did not significantly affect the body weight of the mice in either the CTG-0956 and CTG-1086 patient-derived xenograft models compared to mice treated with an isotype control antibody (FIG. 5B and FIG. 6B).

1. A method of treating ovarian cancer, peritoneal cancer or fallopian tube cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, wherein the antibody-drug conjugate is administered at a dose ranging from about 0.65 mg/kg to about 2.1 mg/kg.

2. The method of claim 1, wherein the dose is about 2.0 mg/kg.

3. The method of claim 1, wherein the dose is 2.0 mg/kg.

4. The method of any one of claims 1-3, wherein the antibody-drug conjugate is administered once about every 3 weeks.

5. The method of any one of claims 1-3, wherein the antibody-drug conjugate is administered once every 3 weeks.

6. The method of claim 1, wherein the dose is about 0.65 mg/kg.

7. The method of claim 1, wherein the dose is 0.65 mg/kg.

8. The method of claim 1, wherein the dose is about 0.9 mg/kg.

9. The method of claim 1, wherein the dose is 0.9 mg/kg.

10. The method of any one of claim 1 or 6-9, wherein the antibody-drug conjugate is administered once about every week.

11. The method of any one of claim 1 or 6-9, wherein the antibody-drug conjugate is administered once every week.

12. The method of any one of claim 1 or 6-9, wherein the antibody-drug conjugate is administered once about every 1 week for 3 consecutive weeks followed by about a 1 week resting period during which the antibody-drug conjugate is not administered.

13. The method of any one of claim 1 or 6-9, wherein the antibody-drug conjugate is administered once every 1 week for three consecutive weeks followed by a one week resting period during which the antibody-drug conjugate is not administered.

14. The method of any one of claim 1 or 6-9, wherein the antibody-drug conjugate is administered on about days 1, 8, and 15 of about a 4-week cycle.

15. The method of any one of claim 1 or 6-9, wherein the antibody-drug conjugate is administered on days 1, 8, and 15 of a 4-week cycle.

16. The method of any one of claims 1-15, wherein the subject has been previously treated with one or more therapeutic agents and did not respond to the treatment, wherein the one or more therapeutic agents is not the antibody-drug conjugate.

17. The method of any one of claims 1-15, wherein the subject has been previously treated with one or more therapeutic agents and relapsed after the treatment, wherein the one or more therapeutic agents is not the antibody-drug conjugate.

18. The method of any one of claims 1-15, wherein the subject has been previously treated with one or more therapeutic agents and has experienced disease progression during treatment, wherein the one or more therapeutic agents is not the antibody-drug conjugate.

19. The method of any one of claims 1-18, wherein the subject has been previously treated with a platinum-based therapy.

20. The method of claim 19, wherein the cancer is platinum-resistant.

21. The method of claim 20, wherein the subject experienced disease progression or relapsed 2 or more months after treatment with the platinum-based therapy.

22. The method of claim 20, wherein the subject experienced disease progression or relapsed within 6 months after treatment with the platinum-based therapy.

23. The method of claim 20, wherein the subject experienced disease progression or relapsed between 2 months and 6 months after treatment with the platinum-based therapy.

24. The method of any one of claims 19-23, wherein cancer is not platinum-refractory.

25. The method of any one of claims 19-24, wherein the subject did not experience disease progression or relapse within 2 months after treatment with the platinum-based therapy.

26. The method of any one of claims 1-25, wherein the subject has been previously treated with a VEGF antagonist.

27. The method of claim 26, wherein the VEGF antagonist is an anti-VEGF antibody.

28. The method of claim 27, wherein the anti-VEGF antibody is bevacizumab.

29. The method of any one of claims 1-28, wherein the subject received prior systemic therapy and experienced disease progression on or after the systemic therapy.

30. The method of any one of claims 1-29, wherein the subject received 1, 2, 3, 4 or 5 rounds of prior systemic therapy.

31. The method of claim 30, wherein the prior systemic therapy is a chemotherapy regimen and wherein poly ADP ribose polymerase (PARP) inhibitors are not chemotherapy.

32. The method of any one of claims 1-31, wherein the cancer is ovarian cancer.

33. The method of claim 32, wherein the ovarian cancer is epithelial ovarian cancer.

34. The method of any one of claims 1-31, wherein the cancer is peritoneal cancer.

35. The method of claim 34, wherein the peritoneal cancer is primary peritoneal cancer.

36. The method of any one of claims 1-31, wherein the cancer is fallopian tube cancer.

37. The method of any one of claims 1-36, wherein the cancer is an advanced stage cancer.

38. The method of claim 37, wherein the advanced stage cancer is a stage 3 or stage 4 cancer.

39. The method of claim 37 or claim 38, wherein the advanced stage cancer is metastatic cancer.

40. The method of any one of claims 1-39, wherein the cancer is recurrent cancer.

41. The method of any one of claims 1-40, wherein the monomethyl auristatin is monomethyl auristatin E (MMAE).

42. The method of any one of claims 1-41, wherein the anti-TF antibody or antigen-binding fragment thereof of the antibody-drug conjugate is a monoclonal antibody or a monoclonal antigen-binding fragment thereof.

43. The method of any one of claims 1-42, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6.

44. The method of any one of claims 1-43, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

45. The method of any one of claims 1-44, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

46. The method of any one of claims 1-45, wherein the anti-TF antibody of the antibody-drug conjugate is tisotumab.

47. The method of any one of claims 1-46, wherein the antibody-drug conjugate further comprises a linker between the anti-TF antibody or antigen-binding fragment thereof and the monomethyl auristatin.

48. The method of claim 47, wherein the linker is a cleavable peptide linker.

49. The method of claim 48, wherein the cleavable peptide linker has a formula: -MC-vc-PAB-, wherein:
a) MC is: 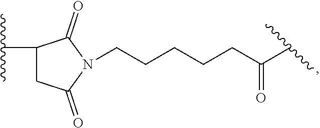 [SMILES: *C(=O)CCCCCN1C(=O)CC(*)C1=O]
b) vc is the dipeptide valine-citrulline, and
c) PAB is:

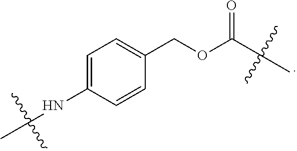 [SMILES: *Nc1ccc(COC(*)=O)cc1]

50. The method of any one of claims 47-49, wherein the linker is attached to sulphydryl residues of the anti-TF antibody obtained by partial reduction or full reduction of the anti-TF antibody or antigen-binding fragment thereof.

51. The method of claim 50, wherein the linker is attached to monomethyl auristatin E (MMAE), wherein the antibody-drug conjugate has the following structure: 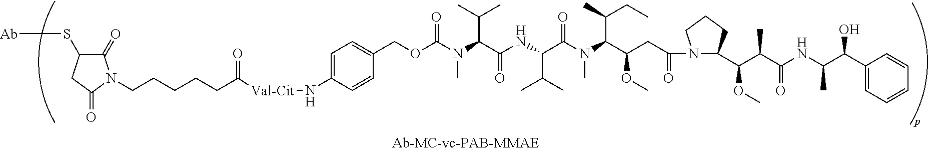
wherein p denotes a number from 1 to 8, S represents a sulphydryl residue of the anti-TF antibody, and Ab designates the anti-TF antibody or antigen-binding fragment thereof.

52. The method of claim 51, wherein the average value of p in a population of the antibody-drug conjugates is about 4.

53. The method of any one of claims 1-52, wherein the antibody-drug conjugate is tisotumab vedotin.

54. The method of any one of claims 1-53, wherein the route of administration for the antibody-drug conjugate is intravenous.

55. The method of any one of claims 1-54, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express TF.

56. The method of any one of claims 1-55, wherein one or more therapeutic effects in the subject is improved after administration of the antibody-drug conjugate relative to a baseline.

57. The method of claim 56, wherein the one or more therapeutic effects is selected from the group consisting of: size of a tumor derived from the cancer, objective response rate, duration of response, time to response, progression free survival, overall survival and CA-125 level.

58. The method of any one of claims 1-57, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.

59. The method of any one of claims 1-58, wherein the objective response rate is at least about 20%, at least about 25%, at least about 30%, at least about 35%, at least about 40%, at least about 45%, at least about 50%, at least about 60%, at least about 70%, or at least about 80%.

60. The method of any one of claims 1-59, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

61. The method of any one of claims 1-60, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

62. The method of any one of claims 1-61, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

63. The method of any one of claims 1-62, wherein the subject exhibits a reduction in CA-125 level in a blood sample from the subject by at least about 10%, at least about 15%, at least about 20%, at least about 25%, at least about 30%, at least about 35%, at least about 40%, at least about 45%, at least about 50%, at least about 60%, at least about 70%, or at least about 80% relative to the CA-125 level in a blood sample obtained from the subject before administration of the antibody-drug conjugate.

64. The method of any one of claims 1-63, wherein the subject has one or more adverse events and is further administered an additional therapeutic agent to eliminate or reduce the severity of the one or more adverse events.

65. The method of any one of claims 1-64, wherein the subject is at risk of developing one or more adverse events and is further administered an additional therapeutic agent to prevent or reduce the severity of the one or more adverse events.

66. The method of claim 64 or claim 65, wherein the one or more adverse events is anaphylaxis, anemia, abdominal pain, hypokalemia, hyponatremia, severe hypersensitivity, epistaxis, an infusion-related reaction, fatigue, nausea, alopecia, conjunctivitis, keratitis, symblepharon, constipation, decreased appetite, diarrhea, vomiting, peripheral neuropathy, or general physical health deterioration.

67. The method of any one of claims 64-66, wherein the one or more adverse events is a grade 3 or greater adverse event.

68. The method of nay one of claims 64-66, wherein the one or more adverse events is a serious adverse event.

69. The method of any one of claims 64-68, wherein the one or more adverse events is conjunctivitis and/or keratitis and the additional agent is a preservative-free lubricating eye drop, an ocular vasoconstrictor and/or a steroid eye drop.

70. The method of any one of claims 1-69, wherein the antibody-drug conjugate is administered as a monotherapy.

71. The method of any one of claims 1-70, wherein the subject is a human.

72. The method of any one of claims 1-71, wherein the antibody-drug conjugate is in a pharmaceutical composition comprising the antibody-drug conjugate and a pharmaceutical acceptable carrier.

73. A kit comprising:
(a) a dosage ranging from about 0.65 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and
(b) instructions for using the antibody drug conjugate according to the method of any one of claims 1-72.


SEQUENCE LISTING (16 sequences)

SEQ ID NO: 1 - protein, 8 aa, Artificial Sequence
  Synthetic Construct
GFTFSNYA

SEQ ID NO: 2 - protein, 8 aa, Artificial Sequence
  Synthetic Construct
ISGSGDYT

SEQ ID NO: 3 - protein, 11 aa, Artificial Sequence
  Synthetic Construct
ARSPWGYYLDS

SEQ ID NO: 4 - protein, 6 aa, Artificial Sequence
  Synthetic Construct
QGISSR

SEQ ID NO: 5 - protein, 3 aa, Artificial Sequence
  Synthetic Construct
AAS

SEQ ID NO: 6 - protein, 9 aa, Artificial Sequence
  Synthetic Construct
QQYNSYPYT

SEQ ID NO: 7 - protein, 118 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, Artificial Sequence
  Synthetic Construct
EVQLLESGGGLVQPGGSLRLSCAAS

SEQ ID NO: 10 - protein, 17 aa, Artificial Sequence
  Synthetic Construct
MSWVRQAPGKGLEWVSS

SEQ ID NO: 11 - protein, 38 aa, Artificial Sequence
  Synthetic Construct
YYTDSVKGRFTISRDNSKNTLYLQMNSLRAEDTAVYYC

SEQ ID NO: 12 - protein, 11 aa, Artificial Sequence
  Synthetic Construct
WGQGTLVTVSS

SEQ ID NO: 13 - protein, 26 aa, Artificial Sequence
  Synthetic Construct
DIQMTQSPPSLSASAGDRVTITCRAS

SEQ ID NO: 14 - protein, 17 aa, Artificial Sequence
  Synthetic Construct
LAWYQQKPEKAPKSLIY

SEQ ID NO: 15 - protein, 36 aa, Artificial Sequence
  Synthetic Construct
SLQSGVPSRFSGSGSGTDFTLTISSLQPEDFATYYC

SEQ ID NO: 16 - protein, 10 aa, Artificial Sequence
  Synthetic Construct
FGQGTKLEIK